(19) United States
(12) Patent Application Publication
(10) Pub. No.: US 2022/0047587 A1
(43) Pub. Date: Feb. 17, 2022

(54) SYNERGISTIC COMPOSITIONS COMPRISING R-2-(SUBSTITUTED-SULFONYL)-HEXAHYDRO-PYRROLO[1,2-A]PYRAZIN-6(2H)-ONES AND S-2-(SUBSTITUTED-SULFONYL)-HEXAHYDRO-PYRROLO[1,2-A]PYRAZIN-6(2H)-ONES IN A NON-RACEMIC RATIO

(71) Applicant: Metys Pharmaceuticals AG, Basel (CH)

(72) Inventors: Carla GHELARDINI, Pistoia (IT); Lorenzo DI CESARE MANNELLI, Prato (IT); Carlo FARINA, Milano (IT); Michael SCHERZ, Oberwil (CH)

(21) Appl. No.: 17/299,930

(22) PCT Filed: Dec. 4, 2019
(86) PCT No.: PCT/EP2019/083599

§ 371 Date: Jun. 4, 2021

(30) Foreign Application Priority Data
Dec. 4, 2018 (EP) 18210128.7

(51) Int. Cl. A61K 31/4985; A61K 45/06; A61P 25/02; A61P 19/02

(52) U.S. Cl. CPC A61K 31/4985; A61P 19/02; A61P 25/02; A61K 45/06

(57) ABSTRACT

The present invention relates to a composition of the enantiomers of 2-(substituted-sulfonyl)-hexahydro-pyrrolo[1,2-a]pyrazin-6(2H)-one derivatives and pharmaceutically acceptable solvates or co-crystals thereof in a certain ratio, a pharmaceutical composition comprising said composition, its use as a medicament and the use of the inventive compositions or pharmaceutical compositions in the treatment and/or prevention of a disease or disorder typically and preferably selected from peripheral sensory neuropathy, preferably peripheral neuropathic pain; seizure; depression; or cognitive impairment.

### FIELD

The present invention relates to compositions and kits comprising R-2-(substituted-sulfonyl)-hexahydro-pyrrolo[1,2-a]pyrazin-6(2H)-ones and S-2-(substituted-sulfonyl)-hexahydro-pyrrolo[1,2-a]pyrazin-6(2H)-ones in a certain range of ratios and pharmaceutically acceptable solvates or co-crystals thereof, pharmaceutical compositions comprising said compositions, their use as a medicament and the uses of the inventive compositions or pharmaceutical compositions or kits for the treatment and/or prevention of a psychiatric disease or disorder typically and preferably selected from depression and treatment-resistant depression, bipolar disorder, post-traumatic stress disorder, obsessive-compulsive disorder, autism spectrum disorder, schizophrenia, and anxiety; acute and chronic central sensitivity disorders such as symptoms of peripheral sensory neuropathy, preferably peripheral neuropathic pain and cold allodynia, fibromyalgia, irritable bowel syndrome, migraine, and cluster headache; and motoneuron disorders such as spinal muscular atrophy, amyotrophic lateral sclerosis, Parkinson's dystonia and Huntington's dystonia.

### BACKGROUND

Glutamic acid is an excitatory neurotransmitter that is widely present in the brain. The first indication of its role as an excitatory messenger emerged in the 1950's, when it was observed that intravenous administration of glutamate induces convulsions. However, the detection of the entire glutamatergic neurotransmitter system, with biosynthetic and catabolic enzymes, cellular uptake mechanisms, intracellular storage and release systems, and its cell-surface ion channels and G protein-coupled receptors, did not take place until the 1970's and 1980's, when suitable pharmacological tools were first identified. It was in the 1990's that the newly emergent tools of molecular biology provided means for the molecular identification and classification of glutamatergic ion channels, receptors, transporters, etc.

The membrane-bound ion channels that are gated by the excitatory amino acids glutamate and glycine, and that also respond to the xenobiotic compound N-methyl-D-aspartate (NMDA), control the flow of both divalent and monovalent cations into pre- and post-synaptic neural cells (see Foster et al., Nature 1987, 329:395-396; Mayer et al., Trends in Pharmacol. Sci. 1990, 11:254-260). They are molecularly, electrophysiologically, and pharmacologically distinct from the glutamate-gated, cation-conducting ion channels that respond to the xenobiotic agents kainate or alpha-amino-3-hydroxy-5-methyl-4-isoxazolepropionic acid (AMPA); and they are similarly distinct from the family of glutamate-gated G protein-coupled receptors, the so-called metabotropic glutamate receptors.

The NMDA-preferring glutamate-gated ion channel has a hetero-tetrameric structural basis: two obligatory GluN1 units and two variable GluN2 receptor subunits encoded by the GRIN1 gene and one of four GRIN2 genes, respectively. One or both GluN2 subunits can be potentially replaced by a GluN3A or a GluN3B subunit. The GRIN1 gene product has 8 splice variants while there are 4 different GRIN2 genes (GRIN2A-D) encoding four distinct GluN2 subunits. The glycine binding site is present on the GluN1 subunit and the glutamate binding site is present on the GluN2 subunit (Paoletti P et al., Nat Rev Neurosci. 2013; 14(6):383-400).

Compounds that modulate NMDA receptor function can be useful in the treatment of many neurological and psychiatric disorders including but not limited to bipolar disorder (Martucci L et al., Schizophrenia Res, 2006; 84(2-3):214-21), major depressive disorder (Li N et al., Biol Psychiatry. 2011; 69(8):754-61), treatment-resistant depression (Preskorn S H et al. J Clin Psychopharmacol. 2008; 28(6):631-7) and other mood disorders (including schizophrenia (Grimwood S et al., Neuroreport. 1999; 10(3):461-5), ante- and postpartum depression (Weickert C S et al. Molecular Psychiatry (2013) 18, 1185-1192), seasonal affective disorder, and the like; Alzheimer's disease (Hanson J E et al., Neurobiol Dis. 2015; 74:254-62; Li S et al., J Neurosci. 2011; 31(18):6627-38) and other dementias (Orgogozo J M et al. Stroke 2002, 33: 1834-1839), Parkinson's disease (Duty S, CNS Drugs. 2012; 26(12):1017-32; Steece-Collier K et al., Exp Neurol. 2000; 163(1):239-43; Leaver K R et al. Clin Exp Pharmacol Physiol. 2008; 35(11):1388-94), Huntington's chorea (Tang T S et al., Proc Natl Acad Sci USA. 2005; 102(7):2602-7; Li L et al., J Neurophysiol. 2004; 92(5):2738-46), multiple sclerosis (Grasselli G et al., Br J Pharmacol. 2013; 168(2):502-17), cognitive impairment (Wang D et al. 2014, Expert Opin Ther Targets 2014; 18(10):1121-30), head injury (Bullock M R et al., Ann N Y Acad Sci. 1999; 890:51-8), spinal cord injury, stroke (Yang Y et al., J Neurosurg. 2003; 98(2):397-403), epilepsy (Naspolini A P et al., Epilepsy Res. 2012 June; 100(1-2):12-9), movement disorders (e.g. dyskinesias) (Morissette M et al., Mov Disord. 2006; 21(1):9-17), various neurodegenerative diseases (e.g. amyotrophic lateral sclerosis (Fuller P I et al., Neurosci Lett. 2006; 399(1-2):157-61) or neurodegeneration associated with bacterial or chronic infections, glaucoma (Naskar R et al. Semin Ophthalmol. 1999 September; 14(3):152-8), pain (e.g. chronic, cancer, post-operative and neuropathic pain (Wu L J and Zhuo M, Neurotherapeutics. 2009; 6(4):693-702), diabetic neuropathy, migraine (Peeters M et al., J Pharmacol Exp Ther. 2007; 321(2):564-72), cerebral ischemia (Yuan H et al., Neuron. 2015; 85(6):1305-18), encephalitis (Dalmau J. et al., Lancet Neurol. 2008; 7(12):1091-8), autism and autism spectrum disorders (Won H. et al., Nature. 2012; 486(7402):261-5), memory and learning disorders (Tang, Y. P. et al., Nature. 1999; 401(6748):63-9), obsessive compulsive disorder (Arnold P D et al., Psychiatry Res. 2009; 172(2):136-9), attention deficit hyperactivity disorder (ADHD) (Dorval K M et al., Genes Brain Behav. 2007; 6(5):444-52), post-traumatic stress disorder (PTSD) (Haller J et al. Behav Pharmacol. 2011; 22(2):113-21; Leaderbrand K et al. Neurobiol Learn Mem. 2014; 113:35-40), tinnitus (Guitton M J, and Dudai Y, Neural Plast. 2007; 80904; Hu S S et al. 2016; 273(2): 325-332), sleep disorders (like narcolepsy or excessive daytime sleepiness, patent WO 2009/058261 A1), vertigo and nystagmus (Straube A. et al., Curr Opitz Neurol. 2005; 18(1):11-4; Starck M et al. J Neurol. 1997 January; 244(1):9-16), anxiety, autoimmunological disorders like neuropsychiatric systemic lupus erythematosus (Kowal C et al. Proc. Natl. Acad. Sci. U.S.A. 2006; 103, 19854-19859) and addictive illnesses (e.g. alcohol addiction, drug addiction) (Nagy J, 2004, Curr Drug Targets CNS Neurol Disord. 2004; 3(3):169-79; Shen H et al., Proc Natl Acad Sci USA. 2011; 108(48):19407-12).

Recent human clinical studies have identified the NMDA-type glutamate-gated ion channel as a novel target of high interest for treatment of depression (Singh J B et al., Biol Psychiatry 2016; 80(6):424-431; Preskorn S H et al. J Clin Psychopharmacol 2008; 28(6):631-7). These studies were conducted using known NMDA-receptor antagonists ketamine and CP-101606, and they have shown significant reductions in depression rating scores in patients suffering with refractory depression. Although, the efficacy was significant, the side effects of using these NDMA receptor antagonists were troublesome.

NMDA-modulating small molecule agonist and antagonist compounds have been developed for potential therapeutic use. However, many of these are associated with very narrow therapeutic indices and undesirable side effects including hallucinations, ataxia, irrational behavior, and significant toxicity, all of which limit their effectiveness and/or safety. Further, 50% or more of patients with depression do not experience an adequate therapeutic response to known administered drugs. In most instances, 2 or more weeks of drug therapy are needed before meaningful improvement is observed, as noted in an open-label study on pharmacological treatment of depression. (Rush et al, Am. J. Psychiatry 2006, 163:1905).

The symptoms of peripheral sensory neuropathy, including one of the most prominent symptoms, peripheral neuropathic pain (Zilliox L A, 2017), are frequently encountered clinical conditions: the prevalence in the general population has been estimated to be between 7% and 10% (van Hecke O et al., 2014). In the United States, painful diabetic peripheral neuropathy alone is estimated to affect approximately 10 million people. Peripheral sensory neuropathy is often resistant to treatment and is associated with poor patient satisfaction of their treatment. Several medications have been shown to be effective in treating peripheral sensory neuropathy associated with diabetic neuropathy and post-herpetic neuralgia, and these medications are often used to treat neuropathic pain associated with other conditions as well. These treatments often have unwanted adverse effects and discontinuation of treatment may be problematic. It is important to recognize that peripheral sensory neuropathy affects many aspects of daily life and is associated with poor general health, reduction in quality of life, poor sleep, and higher anxiety and depression. In fact, measures of quality of life in people with chronic peripheral sensory neuropathy were rated as low as for patients with clinical depression, coronary artery disease, recent myocardial infraction, or poorly controlled diabetes mellitus (Smith B H et al., 2007).

Neuropathic pain medications approved by the US Food and Drug Administration are carbamazepine, duloxetine, pregabalin, gabapentin, topical lidocaine, and topical capsaicin. Tramadol and opioid analgesics are effective in different types of neuropathic pain but are generally not recommended as first-line treatments because of concerns about long-term safety. However, they are recommended as first-line treatments in acute neuropathic pain, neuropathic pain due to cancer, and episodic exacerbations of severe neuropathic pain. The use of strong opioids (codeine, morphine, oxycodone and fentanyl) in the treatment of a variety of neuropathic pain conditions is controversial and a public health concern given the rising number of deaths related to prescription opioids. The serious risks of overdose, dependence, and addiction which these drugs carry may outweigh the potential benefits.

Thus, there remains an urgent and important medical need for the development of novel, orally-effective therapies for the prevention and treatment of neuropathic pain, and for depression and other psychiatric conditions that are toxicologically benign and devoid of the potential for hallucinogenic, dependence and addiction phenomena. There also remains an important medical need for the development of novel, orally-effective therapies for neuropsychiatric diseases, such as those described in the 5th version of the Diagnostic and Statistical Manual of Mental Disorders (DSM-5); and for the treatment of motoneuron diseases, such as amyotrophic lateral sclerosis.

(RS)-2-[(4-fluorophenyl)sulfonyl]hexahydropyrrolo[1,2-a]pyrazin-6-one) is a bicyclic 2-pyrrolidinone derivative with nootropic activity. Racemic 2-[(4-fluorophenyl)sulfonyl]-hexahydropyrrolo[1,2-a]pyrazin-6(2H)-one has more potent nootropic activity than the corresponding single R-enantiomer; and the single R enantiomer has more potent nootropic activity than the corresponding S-enantiomer (Martini et al., Med Chem. 2005, 1(5), pages 473-480). The synthesis of ((RS)-2-[(4-fluorophenyl)sulfonyl]hexahydropyrrolo[1,2-a]pyrazin-6-one) and several derivatives have been described in WO 2009/103176; their stereoselective syntheses are described in Martini et al., Med Chem. 2005, 1(5), pages 473-480. Pharmacological characterization and structure-activity relationship studies are disclosed in Romanelli et al., CNS Drug Rev. 2006, 12(1), pages 39-52; Scapecchi et al., Bioorg Med Chem. 2004, 12(1), pages 71-85; Galeotti et al., Naunyn Schmiedebergs Arch Pharmacol. 2003, 368(6), pages 538-45 and Martini et al., Med Chem. 2005, 1(5), pages 473-480. As a summary of the available reports, it can be said that (RS)-2-[(4-fluorophenyl)sulfonyl]hexahydropyrrolo[1,2-a]pyrazin-6(2H)-one and its R-enantiomer are oral therapies widely effective in rat models of cognition.

As reported in a previous patent application (WO 2009/103176), racemic 2-phenylsulfonyl-hexahydro-pyrrolo[1,2-a]pyrazin-6(2H)-ones also display antihyperalgesic and antiallodynic effects in a wide array of animal models, in which the neuropathic pain is caused by a variety of agents or surgical impairments. In all tested animal models, racemic 2-phenylsulfonyl-hexahydro-pyrrolo[1,2-a]pyrazin-6(2H)-ones show a potency and efficacy profile better than that of standard therapies gabapentin, pregabalin or duloxetine. Furthermore, racemic 2-phenylsulfonyl-hexahydro-pyrrolo[1,2-a]pyrazin-6(2H)-ones show a potency and efficacy profile better than the corresponding single R-enantiomer; and the single R enantiomer is superior to the single S enantiomer. The efficacy of racemic 2-phenylsulfonyl-hexahydro-pyrrolo[1,2-a]pyrazin-6(2H)-ones is evident after a single oral dose. Furthermore, these anti-hyperalgesic and anti-allodynic effects are disease-specific as there is no detectable effect in normal animals; and in an asymmetric surgical model of nerve crush injury, only the injured side of the rat responds to oral administration of the drug.

Dimiracetam is (RS)-3,6,7,7a-tetrahydro-1H-pyrrolo[1,2-a]imidazole-2,5-dione; it is a negative allosteric modulator of spinal NMDA-type glutamate receptors in rat spinal synaptosome preparations (Fariello R G, et al. Neuropharmacology. 2014, 81:85-94) and is orally active in rat models of neuropathic pain, depression, and cognitive impairment (Pinza M, et al. J Med Chem 1993, 36(26):4214-20). The present inventors have surprisingly found that non-racemic mixtures of R and S-dimiracetam lead to activities that are superior to the activities of the single enantiomers and the racemate. This finding is the basis for the co-pending application PCT/EP2018/064125, which is incorporated herein by reference in its entirety.

### SUMMARY

It has now been surprisingly and unexpectedly found that compositions of (R)- and (S)-enantiomers of certain 2-(substituted-sulfonyl)-hexahydro-pyrrolo[1,2-a]pyrazin-6(2H)-ones having an enantiomeric excess (ee) of the (R)-enantiomer greater than or equal to 20% and lower than or equal to 50% exhibit greater pharmacological potency than the corresponding individual enantiomers or even than the racemate and thus provide a synergistic effect that could not have been predicted based on the potency of the individual enantiomers or the racemate. Thus, these non-racemic compositions inhibit NMDA plus glycine-evoked [³H]-D-aspartic acid (used as a mimic of glutamic acid) release from rat brain or spinal synaptosomes more potently than the corresponding individual enantiomers or even than the corresponding racemate. Furthermore, in rodent models of neuropathic pain, cognitive ability, depression, and other models believed to involve glutamate signaling, these non-racemic compositions are more potent than the corresponding individual enantiomers or even than the corresponding racemate.

Thus, the inventive compositions with an enantiomeric excess of (R)-2-(substituted-sulfonyl)-hexahydro-pyrrolo[1,2-a]pyrazin-6(2H)-ones are much more efficient than the corresponding racemic mixture in reducing peripheral neuropathic pain in the paw-pressure test after administration of sodium monoiodoacetate; or in the prevention of oxaliplatin-induced peripheral neuropathic pain; or in the improvement of cognitive function in the passive avoidance step-through assay; or in increasing the swim time in a Porsolt-model of depression.

Therefore, the inventive compositions of (R)- and (S)-enantiomers of 2-(substituted-sulfonyl)-hexahydro-pyrrolo[1,2-a]pyrazin-6(2H)-ones represented by formulae (I) and (II), respectively, having an enantiomeric excess (ee) of the corresponding (R)-enantiomer higher than or equal to 20% and lower than or equal to 50% are pharmacologically more effective at a given dose, as compared to either the pure enantiomers alone or to racemic mixtures of these compounds. The term racemic refers to a 1:1 by weight mixture of (R)- and (S)-enantiomers, which thus has an enantiomeric excess (ee) of 0%. Thus, the effect associated with the present invention is a synergistic effect that surprisingly results from a specific range of ratios between (R)-2-(substituted-sulfonyl)-hexahydro-pyrrolo[1,2-a]pyrazin-6(2H)-ones and (S)-2-(substituted-sulfonyl)-hexahydro-pyrrolo[1,2-a]pyrazin-6(2H)-ones represented by formulae (I) and (II), respectively.

The inventive compositions are beneficial and can be used for the treatment and/or prevention of a psychiatric disease or disorder typically and preferably selected from depression and treatment-resistant depression, bipolar disorder, post-traumatic stress disorder, obsessive-compulsive disorder, autism spec disorder, schizophrenia, and anxiety; acute and chronic central sensitivity disorders such as symptoms of peripheral sensory neuropathy, preferably peripheral neuropathic pain and cold allodynia, fibromyalgia, irritable bowel syndrome, migraine, and cluster headache; and motoneuron disorders such as spinal muscular atrophy, amyotrophic lateral sclerosis, Parkinson's dystonia and Huntington's dystonia.

In a first aspect, the invention provides for a composition comprising a compound of formula (I) (herein also referred to as (R)-2-(substituted-sulfonyl)-hexahydropyrrolo[1,2-a]pyrazin-6(2H)-one) and a compound of formula (II) (herein also referred to as (S)-2-(substituted-sulfonyl)-hexahydropyrrolo[1,2-a]pyrazin-6(2H)-one)

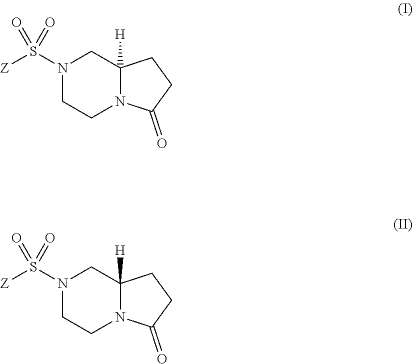

wherein Z is selected from
a straight chain, branched or cyclic C₁₋₄-alkyl group which is optionally substituted with one or more F,
   or
a phenyl group which is substituted with R¹ or R², wherein R¹ is selected from the group consisting of hydrogen, fluoro, chloro, cyano, trifluoromethyl and methyl, and R² is independently selected from the group consisting of hydrogen, fluoro, chloro and methyl;
and R¹ and R² independently occupy any two positions on the phenyl ring;
   and/or pharmaceutically acceptable solvates or co-crystals thereof,
   wherein the enantiomeric excess (ee) of said compound of formula (I) is equal to or higher than 20% and lower than or equal to 50%.

In a preferred aspect, the invention provides for a composition comprising a compound of formula (Ia) (herein also referred to as (R)-2-(phenylsulfonyl)-hexahydropyrrolo[1,2-a]pyrazin-6(2H)-one) and a compound of formula (IIa) (herein also referred to as (S)-2-(phenylsulfonyl)-hexahydropyrrolo[1,2-a]pyrazin-6(2H)-one)

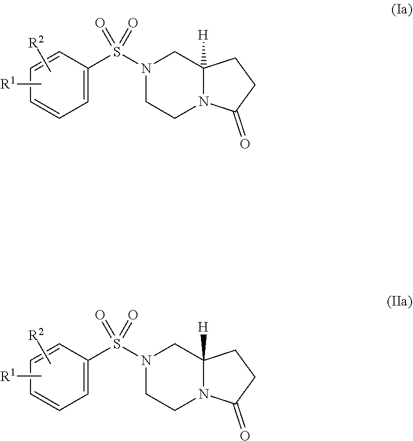

wherein R¹ is selected from the group consisting of hydrogen, fluoro, chloro, cyano, trifluoromethyl and methyl; and R² is independently selected from the group consisting of hydrogen, fluoro, chloro and methyl;
and R¹ and R² independently occupy any two positions on the phenyl ring;
and/or pharmaceutically acceptable solvates or co-crystals thereof,
wherein the enantiomeric excess (ee) of said compound of formula (Ia) is equal to or higher than 20% and lower than or equal to 50%.
In some embodiments of the composition of the present invention, the enantiomeric excess (ee) of said compound of formula (Ia) is equal to or higher than 20% and lower than or equal to 40%.

In a further aspect, the invention provides for a pharmaceutical composition comprising the composition of the present invention and a pharmaceutically acceptable carrier.

In again a further aspect, the invention provides for a kit of parts comprising a compound of formula (I) and a compound of formula (II) and instructions for combining the compound of formula (I) and the compound of formula (II) to obtain an enantiomeric excess (ee) of said compound of formula (I) of equal to or higher than 20% and lower than or equal to 50%. In this aspect, the same preferred ranges of the enantiomeric excess (ee) of said compound of formula (I) and enantiomeric ratios of the compound of formula (I) to the compound of formula (II) as set out herein with respect to the composition apply.

In again a further aspect, the invention provides for the composition of the invention or the pharmaceutical composition of the invention or the kit of the invention for use as a medicament.

In again a further aspect, the invention provides for the composition of the invention or the pharmaceutical composition of the invention or the kit of the invention for the treatment and/or prevention of a psychiatric disease or disorder typically and preferably selected from depression and treatment-resistant depression, bipolar disorder, post-traumatic stress disorder, obsessive-compulsive disorder, autism spectrum disorder, schizophrenia, and anxiety; acute and chronic central sensitivity disorders such as symptoms of peripheral sensory neuropathy, preferably peripheral neuropathic pain and cold allodynia, fibromyalgia, irritable bowel syndrome, migraine, and cluster headache; and motoneuron disorders such as spinal muscular atrophy, amyotrophic lateral sclerosis, Parkinson's dystonia and Huntington's dystonia.

In again a further aspect, the invention provides for a method for the treatment for the treatment and/or prevention of a psychiatric disease or disorder typically and preferably selected from depression and treatment-resistant depression, bipolar disorder, post-traumatic stress disorder, obsessive-compulsive disorder, autism spectrum disorder, schizophrenia, and anxiety; acute and chronic central sensitivity disorders such as symptoms of peripheral sensory neuropathy, preferably peripheral neuropathic pain and cold allodynia, fibromyalgia, irritable bowel syndrome, migraine, and cluster headache; and motoneuron disorders such as spinal muscular atrophy, amyotrophic lateral sclerosis, Parkinson's dystonia and Huntington's dystonia.

In again a further aspect, the invention provides for the use of the composition of the invention or the pharmaceutical composition of the invention or the kit of the invention in the manufacture of a medicament for the treatment and/or prevention of a psychiatric disease or disorder typically and preferably selected from depression and treatment-resistant depression, bipolar disorder, post-traumatic stress disorder, obsessive-compulsive disorder, autism spectrum disorder, schizophrenia, and anxiety; acute and chronic central sensitivity disorders such as symptoms of peripheral sensory neuropathy, preferably peripheral neuropathic pain and cold allodynia, fibromyalgia, irritable bowel syndrome, migraine, and cluster headache; and motoneuron disorders such as spinal muscular atrophy, amyotrophic lateral sclerosis, Parkinson's dystonia and Huntington's dystonia.

In again a further aspect, the invention provides for an article of manufacture comprising the composition of the invention or the pharmaceutical composition of the invention, a container or package and a written description and administration instruction such as a package insert.

Further aspects and embodiments of the present invention will be become apparent as this description continues.

### FIGURES

FIG. 1: Effect on paclitaxel-induced mechanical allodynia of R- and S-enantiomers of 2-(2-fluorobenzenesulfonyl)hexahydropyrrolo[1,2-a]pyrazin-6-one and of different enantiomeric mixtures thereof

FIG. 2: Effect on paclitaxel-induced mechanical hyperalgesia of R- and S-enantiomers of 2-(2-fluorobenzenesulfonyl)hexahydropyrrolo[1,2-a]pyrazin-6-one and of different enantiomeric mixtures thereof

### DETAILED DESCRIPTION

Unless defined otherwise, all technical and scientific terms used herein have the same meanings as commonly understood by one of ordinary skill in the art to which this invention belongs.

The term “about” where used to characterize an enantiomeric excess means ±4% referring to the given numeric value, if not indicated otherwise. In each of the invention embodiments, “about” can be deleted.

The term “preferably” is used to describe features or embodiments which are not required in the present invention but may lead to improved technical effects and are thus desirable but not essential.

A number of compounds are described herein by reference to their structural formula and/or their chemical n e, such as the IUPAC name. In case of discrepancies between the structural formula and the chemical name, the present invention expressly relates to the compounds as referred to by the structural formula as well as by the chemical name.

Any reference herein to the compound or compounds of formula (I) herein is to be understood as also referring to any preferred examples of the compound or compounds of formula (I), such as compounds of formula (Ia). Furthermore, any reference herein to the compound or compounds of formula (II) herein is to be understood as also referring to any preferred examples of the compound or compounds of formula (II), such as compounds of formula (IIa).

The term “treatment” of a disorder or disease as used herein is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease). The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The term “prevention” of a disorder or disease as used herein is also well known in the art. For example, a patient/subject suspected of being prone to suffer from a disorder or disease may particularly benefit from a prevention of the disorder or disease. The subject/patient may undergo a given medical procedure known to carry the risk of developing unwanted effects, such as, for example, the development of peripheral neuropathy symptoms associated with cancer chemotherapy. The subject/patient may have a susceptibility or predisposition or risk factors for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of the aqueous pharmaceutical composition of the present invention before any clinical and/or pathological symptoms are diagnosed or determined or can be diagnosed or determined by the attending physician.

With respect to the numerical values mentioned herein, unless explicitly stated otherwise, the last decimal place of a numerical value preferably indicates its degree of accuracy. Thus, unless other error margins are given, the maximum margin is preferably ascertained by applying the rounding-off convention to the last decimal place. Thus, a value of 2.5 preferably includes the range of 2.45 to 2.54.

The present invention relates to compositions comprising (R)-2-(substituted-sulfonyl)-hexahydropyrrolo[1,2-a]pyrazin-6(2H)-ones of formula (I)) and (S)-2-(substituted-sulfonyl)-hexahydropyrrolo[1,2-a]pyrazin-6(2H)-ones of formula (II) in a certain ratio. It is to be understood that the term “composition” does not require that the pure compound of formula (I) and the pure compound of formula (II) have to be mixed directly. They can be formulated jointly or separately and be administered simultaneously or subsequently, provided that the ratio of the compound of formula (I) and the compound of formula (II) resulting in the subject to be treated is as required by the present invention. Preferably, the inventive composition is a mixture of the compound of formula (I) and the compound of formula (II), but the inventive composition may also encompass a combination of one or more articles containing the compound of formula (I) and one or more articles containing the compound of formula (II), or a combination of one or more articles containing the compound of formula (I) and one or more articles containing a mixture of the compound of formula (I) with the compound of formula (II), e.g. an about 1:1 mixture of the compound of formula (I) and the compound of formula (II), such that the ratio of the compound of formula (I) and compound of formula (II) resulting in the subject to be treated is as required by the present invention.

Furthermore, a 2-(substituted-sulfonyl)-hexahydropyrrolo[1,2-a]pyrazin-6(2H)-one contained in the composition of the present invention has to be present in the overall range of ratios of the compound of formula (I) and the compound of formula (II), alternatively expressed as the enantiomeric excess of the compound of formula (I), required in the present invention. In other words, it is against the gist of the present invention to theoretically split a composition containing equal amounts of the compound of formula (I) and the compound of formula (II) into a component containing an excess of the compound of formula (I) and another component containing an excess of the compound of formula (II). Thus, in whichever physical form the composition of the present invention is, the composition as a whole has to fulfill the requirements regarding the range of ratios of the compound of formula (I) and the compound of formula (II), alternatively expressed as the enantiomeric excess of the compound of formula (I), of the present invention. It is to be understood that the ratios of the compound of formula (I) and the compound of formula (II), alternatively expressed as the enantiomeric excess of the compound of formula (I), are based on a statistically meaningful number of 2-(substituted-sulfonyl)-hexahydropyrrolo[1,2-a]pyrazin-6(2H)-one molecules, which typically exceeds 1000 molecules. In the present invention, the relative amounts of the compound of formula (I) and the compound of formula (II) are expressed either in terms of the ratio of the compound of formula (I) and the compound of formula (II) or in terms of the enantiomeric excess of the compound of formula (I).

It is to be understood that the “ratio” of the compound of formula (I) and the compound of formula (II) as used herein refers to the weight ratio of the compound of formula (I) and the compound of formula (II), unless explicitly stated otherwise. If solvates of the compound of formula (I) and/or the compound of formula (II) are used, the solvent is thus to be disregarded in this calculation. In other words, the “ratio of the compound of formula (I) and the compound of formula (II)” is calculated as follows:

$$Ratio of compound of formula ( I ) and the compound formula ( II ) = \frac{amount of the compound of formula ( I ) by weight}{amount of the compound of formula ( II ) by weight}$$

As known by the skilled person in the art, the ratio of compounds differing only in chirality, such as in the case of the compound of formula (I) and the compound of formula (II), can be determined in a number of ways known in the art, including but not limited to chromatography using a chiral support, capillary electrophoretic separation on a chiral support, polarimetric measurement of the rotation of polarized light, nuclear magnetic resonance spectroscopy using chiral shift reagents, or derivatization of a compound using a chiral compound such as Mosher's acid followed by chromatography or nuclear magnetic resonance spectroscopy. Enantiomers can further be isolated from mixtures by methods known to those skilled in the art, including chiral high-pressure liquid chromatography (HPLC) and direct fractional crystallization of the racemate by chiral co-crystallization techniques, which exploit the formation of specific hydrogen bonding interactions present in co-crystals (see Springuel G R, et al., 2012; and U.S. Pat. No. 6,570,036). Useful co-crystallization partners include enantiomers of mandelic acid, malic acid, tartaric acid and its derivatives; or enantiomers can be prepared by asymmetric syntheses (see, for example, Eliel and Wilen, 1994).

The ratio of the compound of formula (I) and the compound of formula (H) (which may also be referred to as the chiral purity) of the inventive composition such as the non-racemic mixture can also be expressed in terms of its enantiomeric excess (ee), typically and preferably as determined by chiral HPLC (see Examples for details), and calculated by the equation:

ee=(A_{R}−A_{S})/(A_{R}+A_{S})×100%,

wherein A_{R} is the area of the peak of the compound of formula (I), in the HPLC chromatogram of the sample solution and A_{S} is the area of the peak of the compound of formula (II), in the HPLC chromatogram of the sample solution.

In this respect, it is noted that, although chiral “purity” is mentioned above, the gist of the present invention is not achieving a high chiral purity of the compound of formula (I) or the compound of formula (II). Instead, the gist of the present invention is that a certain range of ratios between the compound of formula (I) or the compound of formula (II) leads to a particularly synergistic effect. As opposed to cases in which merely the purity of a compound is to be improved, i.e. where the objective is known, namely one specific compound is to be obtained in a purity of ideally 100%, the present invention is based on a previously unknown ratio of two compounds, namely the compound of formula (I) and the compound of formula (II).

In the present invention, the term “straight chain, branched or cyclic C₁₋₄-alkyl group” refers to any alkyl group having from 1 to 4 carbon atoms. Examples thereof include methyl, ethyl, 1-propyl, 2-propyl, 1-butyl, 2-butyl, iso-butyl and tert-butyl. The term “straight chain, branched or cyclic C₁₋₄-alkyl group which is optionally substituted with one or more F” indicates that the “straight chain, branched or cyclic C₁₋₄-alkyl group” may be substituted with one or more fluorine atoms, which typically replace hydrogen atoms of the alkyl group. The number of fluorine atoms is not particularly limited but is typically from 1 to 10, i.e. 1, 2, 3, 4, 5, 6, 7, 8, 9 or 10. Examples include trifluoromethyl and 1-trifluoromethyl-2,2,2-trifluoroethyl.

The expression “independently occupy any two positions on the phenyl ring” indicates that the group may be attached (or connected) to the phenyl ring at any available position. Typically, the group is attached to the phenyl group replacing a hydrogen atom at that position. Thus, a ring carbon atom of the phenyl ring will be attached to the group (e.g. R¹ or R²) instead of being attached to a hydrogen atom. It is to be understood that neither R¹ nor R² are mandatory substituents. Thus, the phenyl ring may be substituted with either R¹ or R², or both, or none of R¹ and R². R¹ and R² may be attached at any available position, such as ortho, meta or para. Specific examples of substituted phenyl rings include the group consisting of 2-fluorophenyl-, 2-chlorophenyl-, 2-methylphenyl-, 2-cyanophenyl-, 2-trifluoromethylphenyl-, 3-flourophenyl-, 3-chlorophenyl-, 3-methylphenyl-, 3-cyanophenyl-, 3-trifluoromethylphenyl-, 4-fluorophenyl-, 4-chlorophenyl-, 4-methylphenyl-, 4-cyanophenyl-, 4-trifluoromethylphenyl-, 2,3-difluorophenyl-, 2-fluoro-3-chlorophenyl-, 2-fluoro-3-methylphenyl-, 2-fluoro-3-cyanophenyl-, 2-fluoro-3-trifluoromethylphenyl-, 2,4-difluorophenyl-, 2-fluoro-4-chlorophenyl-, 2-fluoro-4-methylphenyl-, 2-fluoro-4-cyanophenyl-, 2-fluoro-5-trifluoromethylphenyl-, 2,5-difluorophenyl-, 2-fluoro-5-chlorophenyl-, 2-chloro-5-methylphenyl-, 2-fluoro-5-cyanophenyl-, 2-fluoro-5-trifluoromethylphenyl-, 2,6-difluorophenyl-, 2-fluoro-6-chlorophenyl-, 2-fluoro-6-methylphenyl-, 2-fluoro-6-cyanophenyl-, 2-fluoro-6-trifluoromethylphenyl-, 2-chloro-3-fluorophenyl-, 2,3-dichlorophenyl-, 2-chloro-3-methylphenyl-, 2-chloro-3-cyanophenyl-, 2-chloro-3-trifluoromethylphenyl-, 2-chloro-4-fluorophenyl-, 2,4-dichlorophenyl-, 2-chloro-4-methylphenyl-, 2-chloro-4-cyanophenyl-, 2-chloro-5-trifluoromethylphenyl-, 2-chloro-5-fluorophenyl-, 2,5-dichlorophenyl-, 2-chloro-5-methylphenyl-, 2-chloro-5-cyanophenyl-, 2-chloro-5-trifluoromethylphenyl-, 2,6-dichlorophenyl-, 2-chloro-6-methylphenyl-, 2-chloro-6-cyanophenyl-, 2-chloro-6-trifluoromethylphenyl-, 2-methyl-3-fluorophenyl-, 2-methyl-3-chlorophenyl-, 2,3-dimethylphenyl-, 2-methyl-3-cyanophenyl-, 2-methyl-3-trifluoromethylphenyl-, 2-methyl-4-fluorophenyl-, 2-methyl-4-chlorophenyl-, 2,4-dimethylphenyl-, 2-methyl-4-cyanophenyl-, 2-methyl-4-trifluoromethylphenyl-, 2-methyl-5-fluorophenyl-, 2-methyl-5-chlorophenyl-, 2,5-dimethylphenyl-, 2-methyl-5-cyanophenyl-, 2-methyl-5-trifluoromethylphenyl-, 2,6-dimethylphenyl-, 2-methyl-6-cyanophenyl-, 2-methyl-6-trifluoromethylphenyl-, 2-cyano-3-fluorophenyl-, 2-cyano-3-chlorophenyl-, 2-cyano-3-methylphenyl-, 2-cyano-4-fluorophenyl-, 2-cyano-4-chlorophenyl-, 2-cyano-4-methylphenyl-, 2-cyano-5-fluorophenyl-, 2-cyano-5-chlorophenyl-, 2-cyano-5-methylphenyl-, 2-trifluoromethyl-3-fluorophenyl 2-trifluoromethyl-3-chlorophenyl-, 2-trifluoromethyl-3-methylphenyl-, 2-trifluoromethyl-4-fluorophenyl-, 2-trifluoromethyl-4-chlorophenyl-, 2-trifluoromethyl-4-methylphenyl-, 2-trifluoromethyl-5-fluorophenyl-, 2-trifluoromethyl-5-chlorophenyl-, 2-trifluoromethyl-5-methylphenyl-, 3,4-difluorophenyl-, 3-fluoro-4-chlorophenyl-, 3-fluoro-4-methylphenyl-, 3-fluoro-4-cyanophenyl-, 3-fluoro-4-trifluoromethylphenyl-, 3-chloro-4-fluorophenyl-, 3,4-dichlorophenyl-, 3-chloro-4-methylphenyl-, 3-chloro-4-cyanophenyl-, 3-chloro-4-trifluoromethylphenyl-, 3-methyl-4-fluorophenyl-, 3-methyl-4-chlorophenyl-, 3,4-dimethylphenyl-, 3-methyl-4-cyanophenyl-, 3-methyl-4-trifluoromethylphenyl-, 3-cyano-4-fluorophenyl-, 3-cyano-4-chlorophenyl-, 3-cyano-4-methylphenyl 3-trifluoromethyl-4-fluorophenyl 3-trifluoromethyl-4-chlorophenyl-, 3-trifluoromethyl-4-methylphenyl-, 3,5-difluorophenyl-, 3-fluoro-5-chlorophenyl-, 3-fluoro-5-methylphenyl 3-fluoro-5-cyanophenyl-, 3-fluoro-5-trifluoromethylphenyl 3,5-dichlorophenyl-, 3-chloro-5-methylphenyl-, 3-chloro-5-cyanophenyl-, 3-chloro-5-trifluoromethylphenyl-, 3,5-dimethylphenyl-, 3-methyl-5-cyanophenyl-, and 3-methyl-5-trifluoromethylphenyl-.

In preferred embodiments, the (substituted) phenyl ring is a group selected from phenyl, 2-fluorophenyl, 3-fluorophenyl and 4-fluorophenyl. More preferably, the (substituted) phenyl ring is a group selected from phenyl, 2-fluorophenyl and 4-fluorophenyl. Even more preferably, the (substituted) phenyl ring is a group selected from phenyl and 2-fluorophenyl.

The term “pharmaceutically acceptable” indicates that the compound or composition, typically and preferably the solvates, co-crystals or carrier, must be compatible chemically or toxicologically with the other ingredient(s), typically and preferably with the inventive composition, when typically and preferably used in a formulation or when typically and preferably used for treating the animal, preferably the human, therewith. Preferably, the term “pharmaceutically acceptable” indicates that the compound or composition, typically and preferably the solvates, co-crystals or carrier, must be compatible chemically and toxicologically with the other ingredient(s), typically and preferably with the inventive composition, when typically and preferably used in a formulation or when typically and preferably used for treating the animal, preferably the human, therewith. It is noted that pharmaceutical compositions can be formulated by techniques known to the person skilled in the art, such as the techniques published in “Remington: The Science and Practice of Pharmacy”, Pharmaceutical Press, 22^{nd} edition.

A “solvate” refers to an association or complex of one or more solvent molecules and either the (R)-enantiomer of formula (I) or the (S)-enantiomer of formula (II). Examples of solvents that form solvates include, but are not limited to, water, isopropanol, ethanol, methanol, dimethyl sulfoxide (DMSO), ethyl acetate, acetic acid, and ethanolamine. The term “hydrate” refers to the complex where the solvent molecule is water.

A “co-crystal” refers to a crystalline structure that contains at least two different compounds that are solid in their pure form under ambient conditions. The at least two different compounds may include the compound of formula (I) and/or the compound of formula (II) and/or any further components of the composition or excipients of the pharmaceutical composition. Co-crystals are made from neutral molecular species, and all species remain neutral after crystallization; further, typically and preferably, they are crystalline homogeneous phase materials where two or more building compounds are present in a defined stoichiometric ratio. See hereto Wang Y and Chen A, 2013; and Springuel G R, et al., 2012; and U.S. Pat. No. 6,570,036. It to be understood that the compounds of formula (I) and/or the compounds of formula (II) may be in the form of any polymorph. A variety of co-crystals and techniques for preparing such co-crystals are described in RSC Drug Discovery, Pharmaceutical Salts and Co-crystals, published in 2012 by the Royal Society of Chemistry and edited by Johan Wouters and Luc Quéré, in particular in chapters 15 and 16. Preferred examples of the co-crystal formers are those disclosed in Table 16.1 of this reference. Even more preferred co-crystals include co-crystals of α-hydroxy acids, α-keto acids and/or α-keto amides with the compounds of formula (I) and (II) in the (R) to (S)-ratios as disclosed herein. Examples of α-hydroxy acids include atrolactic acid, benzilic acid, 4-chloromandelic acid, citric acid, 3,4-dihydroxymandelic acid, ethyl pyruvate, galacturonic acid, gluconolactone, glucuronic acid, glucuronolactone, glycolic acid, 2-hydroxybutanoic acid, 2-hydroxypentanoic acid, 2-hydroxyhexanoic acid, 2-hydroxyheptanoic acid, 2-hydroxyactanoic acid, 2-hydroxynonanoic acid, 2-hydroxydecanoic acid, 2-hydroxyundecanoic acid, 4-hydroxymandelic acid, 3-hydroxy-4-methoxymandelic acid, 4-hydroxy-3-methoxymandelic acid, α-hydroxyarachidonic acid, α-hydroxybutyric acid, α-hydroxyisobutyric acid, α-hydroxylauric acid, α-hydroxymyristic acid, α-hydroxypalmitic acid, α-hydroxystearic acid, 3-(2′-hydroxyphenyl)lactic acid, 3-(4′-hydroxyphenyl)lactic acid, lactic acid, malic acid, mandelic acid, methyllactic acid, methylpyruvate, mucic acid, α-phenylacetic acid, α-phenylpyruvic acid, pyruvic acid, saccharic acid, tartaric acid and tartronic acid. Examples of α-keto acids include 2-ketoethanoic acid (glyoxylic acid), methyl 2-ketoethanoate, 2-ketopropanoic acid (pyruvic acid), methyl 2-ketopropanoate (methyl pyruvate), ethyl 2-ketopropanoate (ethyl pyruvate), propyl 2-ketopropanoate (propyl pyruvate), 2-phenyl-2-ketoethanoic acid (benzoylformic acid), methyl 2-phenyl-2-ketoethanoate (methyl benzoylformate), ethyl 2-phenyl-2-ketoethanoate (ethyl benzoylformate), 3-phenyl-2-ketopropanoic acid (phenylpyruvic acid), methyl 3-phenyl-2-ketopropanoate (methyl phenylpyruvate), ethyl 3-phenyl-2-ketopropanoate (ethyl phenylpyruvate), 2-ketobutanoic acid, 2-ketopentanoic acid, 2-ketohexanoic acid, 2-ketoheptanoic acid, 2-ketooctanoic acid, 2-ketododecanoic acid and methyl 2-ketooctanoate. Examples of α-keto amides include any compounds obtainable by reacting any one of the above examples of α-keto acids with primary or secondary amines.

In a first aspect, the invention provides for a composition comprising a compound of formula (I) and a compound of formula (II)

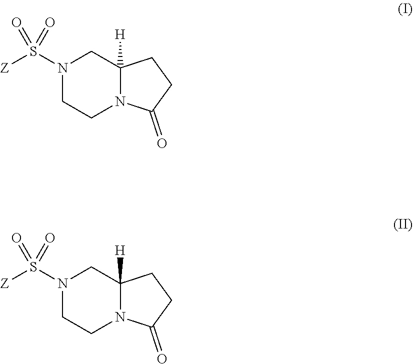

wherein Z is selected from
a straight chain, branched or cyclic C₁₋₄-alkyl group which is optionally substituted with one or more F,
   or
a phenyl group which is substituted with R¹ or R², wherein R¹ is selected from the group consisting of hydrogen, fluoro, chloro, cyano, trifluoromethyl and methyl; and R² is independently selected from the group consisting of hydrogen, fluoro, chloro and methyl;
and R¹ and R² independently occupy any two positions on the phenyl ring;
   and/or pharmaceutically acceptable solvates or co-crystals thereof,
   wherein the enantiomeric excess (ee) of said compound of formula (I) is equal to or higher than 20% and lower than or equal to 50%.

In a preferred aspect, the invention provides for a composition comprising a compound of formula (Ia) and a compound of formula (IIa),

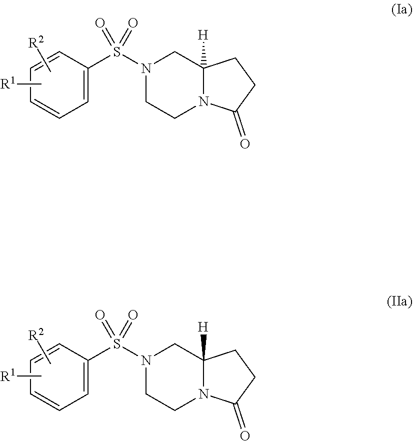

wherein R¹ is selected from the group consisting of hydrogen, fluoro, chloro, cyano, trifluoromethyl and methyl; and R² is independently selected from the group consisting of hydrogen, fluoro, chloro and methyl;
and R¹ and R² independently occupy any two positions on the phenyl ring;
and/or pharmaceutically acceptable solvates or co-crystals thereof,
wherein the enantiomeric excess (ee) of said compound of formula (Ia) is equal to or higher than 20% and lower than or equal to 50%.

Preferably, R¹ is hydrogen, methyl or fluoro. Even more preferably R¹ is fluoro. R² is preferably hydrogen, fluoro or methyl, more preferably hydrogen or fluoro, even more preferably hydrogen.

More preferred compositions are non-racemic mixtures of 2-(2-fluorophenyl)sulfonyl-hexahydropyrrolo[1,2-a]pyrazin-6(2H)-one, non-racemic mixtures of 2-(3-fluorophenyl)sulfonyl-hexahydropyrrolo[1,2-a]pyrazin-6(2H)-one, or non-racemic mixtures of 2-(4-fluorophenyl)sulfonyl-hexahydropyrrolo[1,2-a]pyrazin-6(2H)-one, wherein the enantiomeric excess of the corresponding (R) enantiomer of these more preferred embodiments is equal to or higher than 20% and is lower than or equal to 50%.

These compositions, as well as any other compositions and pharmaceutical compositions according to the present invention, preferably inhibits NMDA plus glycine-evoked [³H]-D-aspartic acid release from rat spinal synaptosomes by at least about 36%, preferably at least about 40%, more preferably at least about 45%, even more preferably about 50%, at a concentration of about 10 nM. An assay for measuring this parameter is set out in Bonanno G et al. Heterocarrier-mediated reciprocal modulation of glutamate and glycine release in rat cerebral cortex and spinal cord synaptosomes. Eur J Pharmacol 1994, 252(1):61-7; and in Fariello R G, Ghelardini C, Di Cesare Mannelli L, Bonanno G, Pittaluga A, Milanese M, Misiano P, Farina C. Broad spectrum and prolonged efficacy of dimiracetam in models of neuropathic pain. Neuropharmacology. 2014 June; 81:85-94. PMID: 24486381.

In the composition according to the present invention, the compound of formula (I) and the compound of formula (II) preferably differ from each other only in the stereochemistry of the stereocenter shown in formulae (I) and (II). In other words, the composition according to the present invention preferably contains the compound of formula (I) and the compound of formula (II) wherein Z is the same in the compound of formula (I) and in the compound of formula (II).

Typically, the non-solvated or non-co-crystallized compositions are preferred. Further preferred are the non-solvated and non-co-crystallized compositions.

Thus, in a further aspect, the invention provides for a composition comprising the compound of formula (I) and the compound of formula (II) in an enantiomeric excess (ee) of the compound of formula (I) of higher than or equal to 20% and lower than or equal to 50%.

More preferably, said enantiomeric excess (ee) of the compound of formula (I) is higher than or equal to 20% and lower than or equal to about 40%. Even more preferably, said enantiomeric excess (ee) of the compound of formula (I) is higher than or equal to 20% and lower than or equal to 35%.

The enantiomeric excess (ee) of the compound of formula (I) is preferably higher than or equal to 22%, 24%, 26%, 28%, 30%, 32%, 34%, 36%, 38% or 40%. On the other hand, the enantiomeric excess (ee) of the compound of formula (I) may preferably be lower than or equal to 48%, 46%, 44%, 42%, 40%, 38%, 36%, 34%, 32% or 30%. Examples of suitable preferred ranges for the enantiomeric excess (ee) of the compound of formula (I) include 20% to 48%, 20% to 46%, 20% to 44%, 20% to 42%, 20% to 40%, 20% to 38%, 20% to 36%, 20% to 34%, 20% to 32%, 20% to 30%, 30% to 50%, 30% to 48%, 30% to 46%, 30% to 44%, 30% to 42%, 30% to 40%, 40% to 50%, 25% to 45%, 25% to 40%, 25% to 35%, 35% to 50%, 35% to 45%, 35% to 40%, etc.

As known to the skilled person, instead of the enantiomeric excess, the ratio of the compound of formula (I) to the compound of formula (II) may be referred to. Preferred ranges for the ratio of the compound of formula (I) to the compound of formula (H) are 1.5:1 to 3.0:1, preferably 1.5:1 to 2.3:1, more preferably 1.5:1 to 2.0:1. Other preferred ranges for the ratio of the compound of formula (I) to the compound of formula (II) are 1.5:1 to 3.3:1, preferably 2:1 to 3:1, more preferably 2:1 or 3:1.

In a further aspect, the invention provides for a pharmaceutical composition comprising the composition of the invention and a pharmaceutically acceptable carrier.

In a further aspect, the invention provides for a kit of parts comprising the compound of formula (I) and the compound of formula (II) and instructions for combining the compound of formula (I) and the compound of formula (II) to obtain an enantiomeric excess (ee) of the compound of formula (I) of equal to or higher than 20% and lower than or equal to 50%. In the following, it is to be understood that the kit according to the present invention may alternatively be used, whenever the use of the composition of the present invention is described. The skilled person will understand that the components of the kit may be combined before administration, which is preferred, or the components of the kit may be administered separately. In the latter case, the components of the kit are typically to be administered within a time range of at most 30 minutes in order to achieve the effects of the present invention.

In again a further aspect, the invention provides for the composition of the invention or the pharmaceutical composition of the invention or the kit of the invention for use as a medicament.

In again a further aspect, the invention provides for the composition of the invention or the pharmaceutical composition of the invention or the kit of the invention for use in the treatment or prevention of a large number of diseases and disorders such as set out in the following:

a) for the prevention or the treatment of positive symptoms of peripheral neuropathy, including cold-sensitivity, tingling, burning, or aching sensations, such as those associated with chemotherapy, antiblastic therapy, viral infection and viral treatment, post-herpetic neuralgia, osteonecrosis, trigeminal neuralgia, or diabetic peripheral neuropathy, to include the primary allodynia, secondary allodynia, or other pains or discomforts associated with sensitization of the spinal cord or higher brain structures or neuronal pathways;
b) for the prevention or the treatment of pain, including bone and joint pain, osteonecrosis pain, repetitive motion pain, dental pain, dysmenorrheal pain, cancer pain, myofascial pain, surgical pain, perioperative pain, and postsurgical pain syndromes such as post-mastectomy syndrome, post-thoracotomy syndrome, or stump pain, as well as pain associated with angina, neuroma pain, complex regional pain syndrome, chronic pelvic pain, chronic lower back pain;
c) for the prevention or the treatment of inflammatory pain, such as osteoarthritis, rheumatoid arthritis, rheumatic disease, chronic arthritic pain and related neuralgias, teno-synovitis and gout;
d) for the prevention or the treatment of neuropathic pain, such as chemotherapy-induced pain, post-traumatic injury pain, crush pain, painful traumatic mononeuropathy, painful polyneuropathy, pain resulting from spinal injury, lumbago, nerve compression or entrapment, sacral pain, trigeminal neuralgia, migraine and migraine headache, post-herpetic neuralgia, phantom limb pain, post-herpetic pain, diabetic neuropathy, central pain syndrome caused a lesion at any level of the peripheral nervous system;
e) for the prevention or the treatment of neuropsychiatric disorders. Examples of neuropsychiatric disorders include schizophrenia, psychosis including schizophrenia, schizophreniform disorder, schizoaffective disorder, delusional disorder, brief psychotic disorder, substance-related disorder, paranoid schizophrenia, disorganized schizophrenia, catatonic schizophrenia or undifferentiated schizophrenia, substance-induced psychotic disorder, substance-related disorders and addictive behaviors;
f) epilepsy and other seizures, both focal and generalized;
g) obesity or other eating disorders associated with excessive food intake, bulimia nervosa;
h) cerebral deficits subsequent to stroke, brain edema, cerebral ischemia, cerebral hemorrhage, neurodegenerative diseases, cardiac bypass surgery and grafting, perinatal hypoxia, cardiac arrest, and hypoglycemic cerebral damage;
i) sleep disorders, such as insomnia, narcolepsy, or restless leg disorder;
j) anxiety disorders, such as affective disorder, panic attacks, panic disorder, acute stress disorder, agoraphobia, generalized anxiety disorder, obsessive-compulsive disorder, post-traumatic stress disorder, separation anxiety disorder, social phobia, specific phobia, substance-induced anxiety disorder;
k) mood disorders, such as depression, anhedonia, unipolar depression, bipolar disorder, psychotic depression;
l) substance addiction, drug dependence, tolerance, dependence or withdrawal from substances including alcohol, amphetamines, cannabis, cocaine, hallucinogens, inhalants, nicotine, opioids, phencyclidine, sedatives, hypnotics or anxiolytics;
m) impaired cognitive function, such as age related cognitive decline or cognitive disorders such as the different types of dementia associated with Alzheimer's disease, ischemia, trauma, vascular problems or stroke, HIV disease, Parkinson's disease, Huntington's disease, Pick's disease, Creutzfeldt-Jacob disease, chemotherapy, perinatal hypoxia, other general medical conditions or substance abuse;
n) Parkinson's disease, including drug-induced parkinsonism, or post-encephalitic parkinsonism;
o) attention deficit disorders, such as attention-deficit hyperactivity disorder (ADHD), obsessive-compulsive disorder, phobia, posttraumatic stress syndrome, autism and autism-spectrum disorders, impulse control disorder;
p) tinnitus, presbycusis;
q) to enhance learning and memory;
r) for the prevention or for the treatment of inherited or sporadic motor neuron disorders. Examples thereof include amyotrophic lateral sclerosis, primary lateral sclerosis, progressive muscular atrophy, progressive bulbar palsy, Friedrich's ataxia, fragile X syndrome;
s) for the prevention or for the treatment of movement disorders. Examples thereof include dystonia, chorea, including Huntington's chorea, Parkinson's-related dystonia, Creutzfeldt-Jakob disease, progressive supranuclear palsy, multiple system atrophy, corticobasal degeneration, basal ganglia calcification;
t) for akinesias such as akinetic-rigid syndromes,
u) for dyskinesias such as medication-induced parkinsonism such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor, including rest tremor, postural tremor and intention tremor, chorea (such as Sydenham's chorea, Huntington's disease, benign hereditary chorea, neuroacanthocytosis, symptomatic chorea, drug-induced chorea and hemiballism), generalized or focal myoclonus, tics (including simple tics, complex tics and symptomatic tics), and dystonia (including generalised dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia), muscular spasms and disorders associated with muscular spasticity or weakness including tremors;
v) and for urinary incontinence, multiple system atrophy, tuberous sclerosis, olivo-ponto-cerebellar atrophy, cerebral palsy, drug-induced optic neuritis, ischemic retinopathy, diabetic retinopathy, glaucoma, spasticity, myoclonus, and Tourette's syndrome-associated dyskinesias.

It is to be understood that the above list of diseases is only given as specific examples and is not to be interpreted as limiting the present invention. Among the above, preferred are one or more selected from a), e), q), r), and s).

The disease or disorder is typically and preferably selected from peripheral sensory neuropathy, preferably peripheral neuropathic pain and other symptoms of peripheral sensory neuropathy; and neuropsychiatric conditions, such as seizure; depression; or cognitive impairment; and motoneuron diseases, such as amyotrophic lateral sclerosis.

Furthermore, the compositions of the present invention can also be used to enhance learning and memory in healthy subjects, e.g. in the form of a non-therapeutic use.

In again a further aspect, the invention provides for a method for the treatment and/or prevention of a disease or disorder, wherein the disease or disorder is typically and preferably selected from peripheral sensory neuropathy, preferably peripheral neuropathic pain and other symptoms of peripheral sensory neuropathy; and neuropsychiatric conditions, such as seizure; depression; or cognitive impairment; and motoneuron diseases, such as amyotrophic lateral sclerosis, wherein said method comprises administration of the composition of the invention or the pharmaceutical composition of the invention or the kit of the invention.

It is also part of the invention to provide a method for the treatment of a disease or disorder, wherein a therapeutically effective amount of the composition of the invention or the pharmaceutical composition of the invention or the kit of the invention is administered to an animal, preferably human, in need thereof. The term “therapeutically effective amount” here refers to that amount sufficient to modulate one or more of the symptoms of the condition or disease being treated, preferably between 10 mg and 3000 mg per administration given once daily or twice daily or three times daily by the oral route. It is furthermore also a part of the invention to provide a method for the prevention of a disease or disorder, wherein a therapeutically effective amount of the composition of the invention or the pharmaceutical composition of the invention or the kit of the invention is administered to an animal, preferably human, reasonably expected to be in need thereof. The term “therapeutically effective amount” here refers to that amount sufficient to modulate one or more of the expected symptoms of the condition or disease to be avoided, preferably between 10 mg and 3000 mg per administration given once daily or twice daily or three times daily by the oral route.

In again a further aspect, the invention provides for the use of the composition of the invention or the pharmaceutical composition of the invention or the kit of the invention in the manufacture of a medicament for use in the treatment and/or prevention of a disease or disorder, wherein the disease or disorder is typically and preferably selected from peripheral sensory neuropathy, preferably peripheral neuropathic pain and other symptoms of peripheral sensory neuropathy; and neuropsychiatric conditions, such as seizure; depression; or cognitive impairment; and motoneuron diseases, such as amyotrophic lateral sclerosis.

It is also part of the present invention to administer the inventive composition or the inventive pharmaceutical composition in association with active principles and active agents, respectively, which present as side effects the insurgence of peripheral neuropathic pain and other symptoms of peripheral neuropathy, in particular with antitumor and antiviral drugs. The composition or the pharmaceutical composition or the kit is preferably used alone or with at least one antitumor drug or at least one antiviral drug. More preferably, the composition or the pharmaceutical composition or the kit is used alone. More preferably, the composition or the pharmaceutical composition or the kit is used with at least one antitumor drug. Alternatively, preferably, the composition or the pharmaceutical composition or the kit is used with at least one antiviral drug.

It is furthermore preferred that the composition or the pharmaceutical composition or the kit is administered in association with at least one antitumor drug or with at least one antiviral drug, wherein said associated administration of said composition or said pharmaceutical composition with said at least one antitumor drug or with said at least one antiviral drug is concurrent, simultaneous, sequential or separate.

Non-limiting examples of such antitumor drugs are selected from the group consisting of a kinase inhibitor, a proteasome inhibitor, a taxane, a vinca alkaloid, and a platinum salt. Non-limiting examples of such antiviral drugs are selected from a nucleoside analog or a nucleotide analog. It is furthermore preferred that said antitumor drug is selected from the group consisting of a kinase inhibitor, a proteasome inhibitor, a taxane, a vinca alkaloid, and a platinum salt. Said antitumor drug is preferably selected from sorafenib, sunitinib, afatinib, axitinib, vandetanib, vemurafenib, ixazomib, bortezomib, paclitaxel, docetaxel, cabazitaxel, vincristine, vinblastine, vindesine, vinorelbine, nedaplatin, lobaplatin, picoplatin, satraplain, cisplatin, carboplatin, and oxaliplatin. Said antiviral drug is preferably selected from zalcitabine, didanosine, stavudine and zidovudine.

The composition or the pharmaceutical composition or the kit is preferably used with at least one antiviral drug, wherein preferably said antiviral drug is selected from a nucleoside or nucleotide, and wherein further preferably said antiviral drug is selected from zalcitabine, didanosine, stavudine or zidovudine.

Said disease or disorder is preferably seizure. Alternatively, said disease or disorder is preferably depression. Further preferably, said disease or disorder is cognitive impairment. Even more preferably, said disease or disorder is peripheral sensory neuropathy. Still more preferably, said disease or disorder is peripheral neuropathic pain.

Said disease or disorder is more preferably peripheral sensory neuropathy, wherein said peripheral sensory neuropathy is selected from the group consisting of (i) diabetic neuropathy, (ii) post-herpetic neuropathy, (iii) lumbago, (iv) sacral pain, (v) surgical pain, (vi) crush injury, (vii) spinal injury, (viii) complex regional pain syndrome, (ix) phantom limb sensations, (x) peripheral sensory neuropathy associated with osteoarthritis, (xi) peripheral sensory neuropathy associated with rheumatoid arthritis, (xii) peripheral sensory neuropathy associated with autoimmune osteoarthrosis, (xiii) cephalea (xiv) fibromyalgia, (xv) peripheral sensory neuropathy induced by antiblastic therapies, (xvi) peripheral sensory neuropathy induced by a chemotherapeutic agent, (xvii) peripheral sensory neuropathy associated with visceral injury, (xviii) peripheral sensory neuropathy associated with osteonecrosis, (xix) peripheral sensory neuropathy associated with human immunodeficiency virus infection and (xx) peripheral sensory neuropathy induced by an antiviral agent.

Said disease or disorder is preferably peripheral sensory neuropathy, wherein said peripheral sensory neuropathy is selected from the gaup consisting of (i) diabetic neuropathy, (ii) post-herpetic neuropathy, (iii) lumbago, (iv) sacral pain, (v) surgical pain, (vi) crush injury, (vii) spinal injury, (viii) complex regional pain syndrome, (ix) phantom limb sensations, (x) peripheral sensory neuropathy associated with osteoarthritis, (xi) peripheral sensory neuropathy associated with rheumatoid arthritis, (xii) peripheral sensory neuropathy associated with autoimmune osteoarthrosis, (xiii) cephalea (xiv) fibromyalgia, (xv) peripheral sensory neuropathy induced by antiblastic therapies, (xvi) peripheral sensory neuropathy induced by a chemotherapeutic agent, (xvii) peripheral sensory neuropathy associated with visceral injury, (xviii) peripheral sensory neuropathy associated with osteonecrosis, (xix) peripheral sensory neuropathy associated with human immunodeficiency virus infection, (xx) peripheral sensory neuropathy induced by an antiviral agent and (xxi) peripheral neuropathic pain.

Said disease or disorder is further preferably peripheral sensory neuropathy, wherein said peripheral sensory neuropathy is diabetic neuropathy. Said disease or disorder is even more preferably peripheral sensory neuropathy, wherein said peripheral sensory neuropathy is post-herpetic neuropathy. Said disease or disorder is preferably peripheral sensory neuropathy, wherein said peripheral sensory neuropathy is lumbago. Said disease or disorder is further preferably peripheral sensory neuropathy, wherein said peripheral sensory neuropathy is sacral pain. Said disease or disorder is further preferably peripheral sensory neuropathy, wherein said peripheral sensory neuropathy is surgical pain. Said disease or disorder is further preferably peripheral sensory neuropathy, wherein said peripheral sensory neuropathy is crush injury. Said disease or disorder is further preferably peripheral sensory neuropathy, wherein said peripheral sensory neuropathy is spinal injury. Said disease or disorder is further preferably peripheral sensory neuropathy, wherein said peripheral sensory neuropathy is complex regional pain syndrome. Said disease or disorder is further preferably peripheral sensory neuropathy, wherein said peripheral sensory neuropathy is phantom limb sensations. Said disease or disorder is further preferably peripheral sensory neuropathy, wherein said peripheral sensory neuropathy is peripheral sensory neuropathy associated with osteoarthritis. Said disease or disorder is further preferably peripheral sensory neuropathy, wherein said peripheral sensory neuropathy is peripheral sensory neuropathy associated with rheumatoid arthritis. Said disease or disorder is further preferably peripheral sensory neuropathy, wherein said peripheral sensory neuropathy is peripheral sensory neuropathy associated with autoimmune osteoarthrosis. Said disease or disorder is further preferably peripheral sensory neuropathy, wherein said peripheral sensory neuropathy is cephalea. Said disease or disorder is further preferably peripheral sensory neuropathy, wherein said peripheral sensory neuropathy is fibromyalgia. Said disease or disorder is still more preferably peripheral sensory neuropathy, wherein said peripheral sensory neuropathy is peripheral sensory neuropathy induced by antiblastic therapies. Said disease or disorder is still more preferably peripheral sensory neuropathy, wherein said peripheral sensory neuropathy is peripheral sensory neuropathy induced by a chemotherapeutic agent. Said disease or disorder is still more preferably peripheral sensory neuropathy, wherein said peripheral sensory neuropathy is peripheral sensory neuropathy associated with visceral injury. Said disease or disorder is still more preferably peripheral sensory neuropathy, wherein said peripheral sensory neuropathy is peripheral sensory neuropathy associated with osteonecrosis. Said disease or disorder is still more preferably peripheral sensory neuropathy, wherein said peripheral sensory neuropathy is peripheral sensory neuropathy associated with human immunodeficiency virus infection. Said disease or disorder is still more preferably peripheral sensory neuropathy, wherein said peripheral sensory neuropathy is peripheral sensory neuropathy induced by an antiviral agent. Said disease or disorder is further preferably peripheral sensory neuropathy, wherein said peripheral sensory neuropathy is peripheral neuropathic pain.

Said peripheral sensory neuropathy is preferably selected from peripheral sensory neuropathy induced by a chemotherapeutic agent or peripheral sensory neuropathy induced by an antiviral agent.

Said disease or disorder is still more preferably peripheral sensory neuropathy induced by a chemotherapeutic agent, wherein typically and preferably said chemotherapeutic agent is selected from the group consisting of a kinase inhibitor, a proteasome inhibitor, a taxane, a vinca alkaloid and a platinum salt. Still more preferably, said disease or disorder is peripheral sensory neuropathy induced by a chemotherapeutic agent, wherein said chemotherapeutic agent is selected from the group consisting of a kinase inhibitor, a proteasome inhibitor, a taxane, a vinca alkaloid and a platinum salt. Still more preferably, said disease or disorder is peripheral sensory neuropathy induced by a chemotherapeutic agent, wherein said chemotherapeutic agent is selected from the group consisting of sorafenib, sunitinib, afatinib, axitinib, vandetanib, vemurafenib, ixazomib, bortezomib, paclitaxel, docetaxel, cabazitaxel, vincristine, vinblastine, vindesine, vinorelbine, nedaplatin, lobaplatin, picoplatin, satraplain, cisplatin, carboplatin, or oxaliplatin. Said disease or disorder is still more preferably peripheral sensory neuropathy induced by a chemotherapeutic agent, wherein said chemotherapeutic agent is selected from the group consisting of sorafenib, vincristine, paclitaxel, or oxaliplatin. Very preferably, said peripheral sensory neuropathy is induced by a chemotherapeutic agent, wherein said chemotherapeutic agent is sorafenib, paclitaxel, vincristine, cisplatin, carboplatin or oxaliplatin.

Further preferably, said disease or disorder is peripheral sensory neuropathy induced by an antiviral agent, wherein preferably said antiviral agent is a nucleoside reverse transcriptase inhibitor. Still more preferably, said disease or disorder is peripheral sensory neuropathy induced by an antiviral agent, wherein said antiviral agent is selected from zalcitabine, didanosine, stavudine or zidovudine. Still more preferably, said disease or disorder is peripheral sensory neuropathy induced by zalcitabine.

Preferably, said chemotherapy-induced peripheral sensory neuropathy entails symptoms of allodynia or dysesthesia, more preferably allodynia or dysesthesia of the hands or feet, and further preferably allodynia or dysesthesia of the hands or feet induced by sorafenib, by vincristine, by paclitaxel, or by carboplatin, cisplatin, or oxaliplatin.

Still more preferably, said peripheral sensory neuropathy is associated with pain, paresthesias, dysesthesias or allodynia.

Further preferably, the inventive composition or the inventive pharmaceutical composition may be administered prophylactically, starting before the antitumoral chemotherapeutic principle has induced peripheral sensory neuropathy and its attendant symptoms.

Further preferably, the inventive composition or the inventive pharmaceutical composition may be administered intermittently. Furthermore it is a preferred in the present invention that the inventive composition or the inventive pharmaceutical composition may be administered in synchrony with repeated cycles of an antitumoral chemotherapeutic principle.

The dosage will depend on the route of administration, the severity of the disease, age and weight of the patient or subject and other factors normally considered by the attending physician, when determining the individual regimen and dosage level for a particular patient or subject.

The composition or pharmaceutical composition of the invention may be administered via any route, including oral, intramuscular, subcutaneous, topical, transdermal, intranasal, intravenous, sublingual or intrarectal administration. Typically and preferably, the pharmaceutical composition of the invention is administered in a single dosage unit once-daily, twice-daily or three times-daily via the oral route, and most preferably once-daily or twice-daily. In the most preferred embodiment, the composition or pharmaceutical composition of the invention is administered twice daily.

Typically and preferably, the oral dose of the inventive composition or the inventive pharmaceutical composition is between 10 mg and 3000 mg per administration, more preferably between 20 mg to 2000 mg per administration, again more preferably between 50 mg and 1000 mg per administration. Typically and preferably, said composition or said pharmaceutical composition is administered orally twice daily in a dose of between 10 mg and 3000 mg per administration, more preferably between 20 mg to 2000 mg per administration, again more preferably between 50 mg and 1000 mg per administration.

The pharmaceutical composition of the invention may be prepared by mixing suitably selected and pharmaceutically acceptable excipients, vehicles, adjuvants, additives, surfactants, desiccants or diluents known to those well-skilled in the art, and can be suitably adapted for oral, parenteral or topical administration. Typically and preferably the pharmaceutical composition of the invention is administered in the form of a tablet, capsule, sachets, powder, granule, pellet, oral or parenteral solution, suspension, suppository, ointment, cream, lotion, gel, paste and/or may contain liposomes, micelles and/or microspheres.

The pharmaceutically acceptable carrier of the pharmaceutical composition of the invention is without limitation any pharmaceutically acceptable excipient, vehicle, adjuvant, additive, surfactant, desiccant or diluent. Suitable pharmaceutically acceptable carriers are magnesium carbonate, magnesium stearate, talc, lactose, sugar, pectin, dextrin, starch, tragacanth, methyl cellulose, sodium carboxymethyl cellulose, a low-melting wax, cocoa butter. Pharmaceutically acceptable carriers of the invention can be solid, semi-solid or liquid.

Tablets, capsules or sachets for oral administration are usually supplied in dosage units and may contain conventional excipients, such as binders, fillers, diluents, tableting agents, lubricants, detergents, disintegrants, colorants, flavors and wetting agents. Tablets may be coated in accordance to methods well known in the art. Suitable fillers include or are preferably cellulose, mannitol, lactose and similar agents. Suitable disintegrants include or are preferably starch, polyvinyl pyrrolidone and starch derivatives such as sodium starch glycolate. Suitable lubricants include or are preferably, for example, magnesium stearate. Suitable wetting agents include or are preferably sodium lauryl sulfate. These solid oral compositions can be prepared with conventional mixing, filling or tableting methods. The mixing operations can be repeated to disperse the active agent in compositions containing large quantities of fillers. These operations are conventional.

The oral liquid compositions can be provided in the form of, for example, aqueous solutions, emulsions, syrups or elixirs or in the form of a dry product to be reconstituted with water or with a suitable liquid carrier at the time of use. The liquid compositions can contain conventional additives, such as suspending agents, for example sorbitol, syrup, methylcellulose, gelatin, hydroxyethyl cellulose, carboxymethyl cellulose, aluminum stearate gel or hydrogenated edible fats; emulsifying agents, for example lecithin, sorbitan monooleate, or acacia; non aqueous carriers (which can include edible oil), for example almond oil, fractionated coconut oil, oily esters, such as glycerin esters, propylene glycol or ethyl alcohol; preservatives, for example methyl or propyl p-hydroxybenzoate or sorbic acid and if desired, conventional flavors or colorants. Oral formulations may also include or may be formulated as conventional formulations, such as tablets or granules. For parenteral administration, liquid dosage units can be prepared containing the inventive composition and a sterile carrier.

Oral formulations may optionally further include taste-masking components to optimize the taste perception of the oral formulation. Examples of such taste-masking components may be citrusphenyl-, licoricephenyl-, mintphenyl-, grapephenyl-, black currant- or eucalyptus-based flavorants known to those well-skilled in the art.

The parenteral solutions are normally prepared by dissolving the compound in a carrier and sterilizing by filtration, before filling suitable vials or ampoules and sealing.

Adjuvants, such as local anesthetics, preservatives and buffering agents can be added to the pharmaceutical composition. In order to increase stability, the composition can be frozen after filling the vial and the water removed under vacuum. A surfactant or humectant can be advantageously included in the pharmaceutical composition in order to facilitate uniform distribution of the inventive composition.

Topical formulations include or are preferably ointments, creams, lotions, gels, gums, solutions, pastes or may contain liposomes, micelles or microspheres.

Subjects to be treated by the composition or pharmaceutical composition of the invention are humans and animals. Preferred animals are domestic and farm animals, including but not limited to guinea pig, rabbit, horse, donkey, camel, cow, sheep, goat, pig, cat, dog and parrot. More preferred subjects are mammals, again more preferably humans.

In again a further aspect, the invention provides for an article of manufacture comprising the composition of the invention or the pharmaceutical composition of the invention or the kit of the invention, a container or package and a written description and administration instruction such as a package insert.

It is further envisaged that compositions of the compound of formula (I), or the compound of formula (H) with racetams such as aniracetam, brivaracetam, cebaracetam, coluracetam, dimiracetam, doliracetam, dupracetam, etiracetam/levetiracetam, fasoracetam, imuracetam, methylphenylpiracetam, nebracetam, nefiracetam, omberacetam (Noopept), oxiracetam, phenylpiracetam, phenylpiracetam hydrazide, piracetam, pramiracetam, rolipram, rolziracetam and/or seletracetam may also be used to prepare synergistic mixtures and compositions, in particular if the ratio of the compound of formula (I), or the compound of formula (II), and the racetam, or an enantiomer of the other racetam, are chosen within the ranges disclosed herein for the mixtures of the compound of formula (I) and the compound of formula (II).

It is further envisaged that compositions of the compound of formula (I), or the compound of formula (II) with other compounds, such as dimiracetam or dimiracetam-like compounds disclosed in U.S. Pat. No. 7,544,705 or in U.S. Pat. No. 8,334,286, may also be used to prepare synergistic mixtures and compositions, in particular if the ratio of the compound of formula (II) (or (I)), and the dimiracetam-like compound or an enantiomer of a dimiracetam-like compound, are chosen within the ranges disclosed herein for the mixtures of the compound of formula (I), and the compound of formula (II).

The present invention also relates to a method of treating and/or preventing a disease, injury, or disorder, comprising: administering to a subject the composition of claim 1, wherein the disease, injury, or disorder is peripheral sensory neuropathy, seizure, depression, or cognitive impairment. In this method, the disease, injury, or disorder is preferably peripheral sensory neuropathy, a neuropsychiatric disorder, a motoneuron disorder, or a movement disorder. More preferably, the disease, injury, or disorder is peripheral sensory neuropathy. The peripheral sensory neuropathy is preferably peripheral neuropathic pain. The peripheral sensory neuropathy is preferably selected from diabetic neuropathy, post-herpetic neuropathy, lumbago, sacral pain, surgical pain, crush injury, spinal injury, complex regional pain syndrome, phantom limb sensations, peripheral sensory neuropathy associated with osteoarthritis, peripheral sensory neuropathy associated with rheumatoid arthritis, peripheral sensory neuropathy associated with autoimmune osteoarthrosis, cephalea, fibromyalgia, peripheral sensory neuropathy induced by antiblastic therapies, peripheral sensory neuropathy induced by a chemotherapeutic agent, peripheral sensory neuropathy associated with visceral injury, peripheral sensory neuropathy associated with osteonecrosis, peripheral sensory neuropathy associated with human immunodeficiency virus infection, peripheral neuropathic pain, or peripheral sensory neuropathy induced by an antiviral agent. In some instances, the peripheral sensory neuropathy is peripheral sensory neuropathy induced by a chemotherapeutic agent or peripheral sensory neuropathy induced by an antiviral agent. In certain instances, the peripheral sensory neuropathy is peripheral sensory neuropathy induced by a chemotherapeutic agent, wherein the chemotherapeutic agent is selected from the group consisting of a kinase inhibitor, a proteasome inhibitor, a taxane, a vinca alkaloid, and a platinum salt, and wherein preferably the chemotherapeutic agent is selected from sorafenib, sunitinib, afatinib, axitinib, vandetanib, vemurafenib, ixazomib, bortezomib, paclitaxel, docetaxel, cabazitaxel, vincristine, vinblastine, vindesine, vinorelbine, nedaplatin, lobaplatin, picoplatin, satraplain, cisplatin, carboplatin, and oxaliplatin. In some instances, the peripheral sensory neuropathy is peripheral sensory neuropathy induced by an antiviral agent, wherein the antiviral agent is a nucleoside reverse transcriptase inhibitor. In some instances, the nucleoside reverse transcriptase inhibitor is zalcitabine, didanosine, stavudine, or zidovudine.

In some instances, the method further comprises administering an antitumor drug, wherein the antitumor drug is selected from the group consisting of a kinase inhibitor, a proteasome inhibitor, a taxane, a vinca alkaloid, and a platinum salt. In some instances, the antitumor drug is selected from the group consisting of sorafenib, sunitinib, afatinib, axitinib, vandetanib, vemurafenib, ixazomib, bortezomib, paclitaxel, docetaxel, cabazitaxel, vincristine, vinblastine, vindesine, vinorelbine, nedaplatin, lobaplatin, picoplatin, satraplain, cisplatin, carboplatin, and oxaliplatin. In some instances, the method further comprises administering an antiviral drug, wherein the antiviral drug is a nucleoside or a nucleotide. In some instances, the antiviral drug is zalcitabine, didanosine, stavudine, or zidovudine. In some instances, the composition is administered orally twice daily in a dose of between 10 mg and 3000 mg per administration, between 20 mg to 2000 mg per administration, or between 50 mg and 1000 mg per administration.

The present invention furthermore relates to a method of enhancing learning and memory, comprising administering to a subject the composition of the present invention as described herein. In some instances of this method, the subject is a healthy subject.

The non-patent references cited herein are abbreviated by first author accompanied by the year of publication. The complete citations are listed in the following.
Attal N, Cruccu G, Baron R, Haanpää M, Hansson P, Jensen T S, Nurmikko T; European Federation of Neurological Societies. EFNS guidelines on the pharmacological treatment of neuropathic pain: 2010 revision. Eur J Neural. 2010 September; 17(9):1113-e88. PMID: 20402746
Bradford M M. A rapid and sensitive method for the quantitation of microgram quantities of protein utilizing the principle of protein-dye binding. Anal Biochem. 1976 May 7; 72:248-54. PMID: 942051
Bril V, England J, Franklin G M, Backonja M, Cohen J, Del Toro D, Feldman E, Iverson D J, Perkins B, Russell J W, Zochodne D; American Academy of Neurology; American Association of Neuromuscular and Electrodiagnostic Medicine; American Academy of Physical Medicine and Rehabilitation. Evidence-based guideline: Treatment of painful diabetic neuropathy: report of the American Academy of Neurology, the American Association of Neuromuscular and Electrodiagnostic Medicine, and the American Academy of Physical Medicine and Rehabilitation. Neurology. 2011 May 17; 76(20):1758-65. Erratum in: Neurology. 2011 Aug. 9; 77(6):603. PMID: 21482920
Camilleri P, Eggleston D, Farina C, Murphy J A, Pfeiffer U, Pinza M, Senior L A. Chiral high-performance liquid chromatography of some related bicyclic lactams. Journal of Chromatography A, 654 (1993) 207-213.
Cavaletti G, Tredici G, Petruccioli M G, Dondè E, Tredici P, Marmiroli P, Minoia C, Ronchi A, Bayssas M, Etienne G G. Effects of different schedules of oxaliplatin treatment on the peripheral nervous system of the rat. Eur J Cancer. 2001 December; 37(18):2457-63. PMID: 11720843
Christensen D, Idänpään-Heiklilâ J J, Guilbaud G, Kayser V. The antinociceptive effect of combined systemic administration of morphine and the glycine/NMDA receptor antagonist, (+)-HA966 in a rat model of peripheral neuropathy. Br J Pharmacol. 1998 December; 125(8):1641-50. Erratum in: Br J Pharmacol 1999 April; 126(8):1881. PMID: 9886755
Dubinsky R M, Kabbani H, El-Chami Z, Boutwell C, Ali H; Quality Standards Subcommittee of the American Academy of Neurology. Practice parameter: treatment of postherpetic neuralgia: an evidence-based report of the Quality Standards Subcommittee of the American Academy of Neurology. Neurology. 2004 Sep. 28; 63(6):959-65. PMID: 15452284
Di Cesare Mannelli L, Maresca M, Farina C, Scherz M W, Ghelardini C. A model of neuropathic pain induced by sorafenib in the rat: Effect of unifiram. Neurotoxicology. 2015a September; 50:101-7. PMID: 26254739
Di Cesare Mannelli L, Micheli L, Farina C, Scherz M, and Ghelardini C. Effects of unifiram on oxaliplatin-induced hyperalgesia and allodynia in the rat. Journal of Clinical Oncology 2015b 33:15_suppl, e20650-e20650.
Eliel E L, Wilen S H. Stereochemistry of Organic Compounds, Wiley-Interscience, New York (1994). ISBN: 978-0-471-01670-0
Farina C, Gagliardi S, Ghelardini C, Martinelli M, Norcini M, Parini C, Petrillo P, Ronzoni S. Bioorg Med Chem. 2008; 16(6):3224-32. PMID: 18171618
Fariello R G, Ghelardini C, Di Cesare Mannelli L, Bonanno G, Pittaluga A, Milanese M, Misiano P, Farina C. Broad spectrum and prolonged efficacy of unifiram in models of neuropathic pain. Neuropharmacology. 2014 June; 81:85-94. PMID: 24486381
Fariello R G, Ghelardini C, Di Cesare Manneli L, Zanardelli M, Farina C. Antidepressant-like activity of unifiram (NT-11624) in the rat forced swimming test. Program No. 789.08/EE25. 2011 Neuroscience Meeting Planner. Washington, D.C.: Society for Neuroscience, 2011. Online.
Fernihough J, Gentry C, Malcangio M, Fox A, Rediske J, Pellas T, Kidd B, Bevan S, Winter J. Pain related behaviour in two models of osteoarthritis in the rat knee. Pain. 2004; November; 112(1-2):83-93. PMID: 15494188
Finnerup N B, Attal N, Haroutounian S, McNicol E, Baron R, Dworkin R H, Gilron I, Haanpää M, Hansson P, Jensen T S, Kamerman P R, Lund K, Moore A, Raja S N, Rice A S, Rowbotham M, Sena E, Siddall P, Smith B H, Wallace M. Pharmacotherapy for neuropathic pain in adults: a systematic review and meta-analysis. Lancet Neurol. 2015 February; 14(2):162-73. PMID: 25575710
Gronseth G, Cruccu G, Alksne J, Argoff C, Brainin M, Burchiel K, Nurmikko T, Zakrzewska J M. Practice parameter: the diagnostic evaluation and treatment of trigeminal neuralgia (an evidence-based review): report of the Quality Standards Subcommittee of the American Academy of Neurology and the European Federation of Neurological Societies. Neurology. 2008 Oct. 7; 71(15):1183-90. PMID: 18716236
Guingamp C, Gegout-Pottie P, Philippe L, Terlain B, Netter P, Gillet P. Mono-iodoacetate-induced experimental osteoarthritis: a dose-response study of loss of mobility, morphology, and biochemistry. Arthritis Rheum. 1997 September; 40(9):1670-9. PMID: 9324022
Guzman R E, Evans M G, Bove S, Morenko B, Kilgore K. Mono-iodoacetate-induced histologic changes in subchondral bone and articular cartilage of rat femorotibial joints: an animal model of osteoarthritis. Toxicol Pathol. 2003; 31(6):619-24. PMID: 14585729
Latremoliere A, Woolf C J. Central sensitization: a generator of pain hypersensitivity by central neural plasticity. J Pain. 2009 September; 10(9):895-926. PMID: 19712899.
Hansen K B, Yi F, Perszyk E, Furukawa H, Wollmuth L P, Gibb A J, Traynelis S F. Structure, function, and allosteric modulation of NMDA receptors. J Gen Physiol 2018, 150:8, 1081-1105.
Manetti D, Ghelardini C, Bartolini et al. Design, synthesis and preliminary pharmacological evaluation of 1,4-diazabicyclo[4.3.0]nonan-9-ones as a new class of highly potent nootropic drugs. J Med Chem 2000; 43:1969-1974.
Martini E, Ghelardini C, Bertucci C, et al. Enantioselective synthesis and preliminary pharmacological evaluation of the enantiomers of unifiram (DM232), a potent cognition-enhancing agent. Med Chem 2005; 1:473-480.
Marieb E N, Wilhelm P B & Mallat J B, eds. Human Anatomy, 8^{th} edition. Pearson, London (2017).
Moulin D, Boulanger A, Clark A J, Clarke H, Dao T, Finley G A, Furlan A, Gilron I, Gordon A, Morley-Forster P K, Sessle B J, Squire P, Stinson J, Taenzer P, Velly A, Ware M A, Weinberg E L, Williamson O D; Canadian Pain Society. Pharmacological management of chronic neuropathic pain: revised consensus statement from the Canadian Pain Society. Pain Res Manag. 2014 November-December; 19(6):328-35. PMID: 25479151
Nakamura Y, Iga K, Shibata T, Shudo M, Kataoka K. Glial plasmalemmal vesicles: a subcellular fraction from rat hippocampal homogenate distinct from synaptosomes. Glia. 1993 September; 9(1):48-56. PMID: 7902337
Paluzzi S, Alloisio S, Zappettini S, Milanese M, Raiteri L, Nobile M, Bonanno G. Adult astroglia is competent for Na+/Ca2+ exchanger-operated exocytotic glutamate release triggered by mild depolarization. J Neurochem. 2007 November; 103(3):1196-207. PMID: 17935604
Pinza M, Farina C, Cerri A, Pfeiffer U, Riccaboni M T, Banfi S, Biagetti R, Pozzi O, Magnani M, Dorigotti L. Synthesis and pharmacological activity of a series of dihydro-1H-pyrrolo[1,2-a]imidazole-2,5(3H,6H)-diones, a novel class of potent cognition enhancers. J Med Chem. 1993 Dec. 24; 36(26):4214-20. PMID: 8277504
Randall L O, Selito J J. A method for measurement of analgesic activity on inflamed tissue. Arch Int Pharmacodyn Ther. 1957 Sep. 1; 111(4):409-19. PMID: 13471093
Scapecchi S, Martini E, Manetti D, et al. Structure-activity relationship studies on unifiram (DM232) and sunifiram (DM235), two novel and potent cognition enhancing drugs. Bioorg Med Chem 2004; 12:71-85.
Smith B H, Torrance N, Bennett M I, Lee A J. Health and quality of life associated with chronic pain of predominantly neuropathic origin in the community. Clin J Pain. 2007 February; 23(2):143-9. PMID: 17237663
Springuel G R, Leyssens T. Innovative chiral resolution using enantiospecific co-crystallization in solution. Cryst Growth Des. 2012; 12 (7): 3374-3378. DOI: 10.1021/cg300307z.
Torchio L, Lombardi F, Visconti M, Doyle E. Determination of the polar drug unifiram in human plasma and serum by column-switching high-performance liquid chromatography. J Chromatogr B Biomed Appl. 1995 Apr. 7; 666(1):169-77. PMID: 7655615
van Hecke O, Austin S K, Khan R A, Smith B H, Torrance N. Neuropathic pain in the general population: a systematic review of epidemiological studies. Pain. 2014 April; 155(4):654-62. Erratum in: Pain. 2014 September; 155(9):1907. PMID: 24291734
Wang Y, Chen A. Crystallization-based separation of enantiomers, in Stereoselective Synthesis of Drugs and Natural Products, 2^{nd} volume; Andrushko V, Andrushko N, Eds, Wiley-Interscience, New York (2013). ISBN: 978-1-118-03217-6
Zilliox L A. Neuropathic Pain. Continuum (Minneap Minn). 2017 April; 23(2, Selected Topics in Outpatient Neurology):512-532. PMID: 28375916.

### EXAMPLES

Examples of the present invention are purely for illustrative and non-limiting purposes. Samples of racemic 2-phenylsulfonyl-hexahydro-pyrrolo[1,2-a]pyrazin-6(2H)-ones and derivatives as well as the individual enantiomers of formulae (I) and (II) can be synthesized using commercially available starting materials, such as purchased from Sigma-Aldrich. These commercial supplies can be used as received from the supplier without further purification, using methods and techniques of preparative synthesis well known to those skilled in the art.

### Example 1: Synthesis of 2-phenylsulfonyl-hexahydro-pyrrolol[1,2-a]pyrazin-6(2M-ones

(R)-enantiomers, (S)-enantiomers and racemic mixtures of 2-phenylsulfonyl-hexahydro-pyrrolo[1,2-a]pyrazin-6(2H)-ones were prepared in accordance with methods described in Manetti D, et al. 2000; Martini E, et al. 2005; and in Scapecchi S, et al. 2004. The enantiomeric excess of the synthesized (R)- and (S)-derivatives was determined as described in Manetti D, et al. 2000. The enantiomeric excess of (R)- and (S)-enantiomers, when used separately for preparing the composition of the present invention, is equal to or greater than 96% for each enantiomer.

For achieving the desired enantiomeric excess of equal to or higher than 20% ee (excess (R)) and less than or equal to 50% ee (excess (R)), as well as other desired specific compositions in accordance with the present invention, several methods known to the skilled person in the art can be applied. For example, said compositions are prepared either by mixing the individual enantiomers or by mixing the racemate of a 2-phenylsulfonyl-hexahydro-pyrrolo[1,2-a]pyrazin-6(2H)-one with the respective quantities of the (R)-enantiomer. Furthermore, starting from a racemic 2-phenylsulfonyl-hexahydro-pyrrolo[1,2-a]pyrazin-6(2H)-one, part or all of the (5)-enantiomer may be removed by preparative chiral column chromatography.

### Example 2: Rat Models of Induced Peripheral Neuropathic Pain

### Evaluation of Pain Responses

At the peak of the pain response according to the model under evaluation, the effects of a single dose of the test compounds, vehicles and comparators were evaluated. Thereafter, to assess the possible development of tolerance, repeated administrations of mixtures of the 2-phenylsulfonyl-hexahydro-pyrrolo[1,2-a]pyrazin-6(2H)-ones or derivatives were studied. Both hyperalgesia and allodynia were assessed. All efficacy evaluations were carried out by investigators blinded to the rats' treatment allocation.

Results are expressed as the minimum dose (via the indicated route) at which a statistically-significant difference is observed when compared to time-matched injured rats receiving a vehicle administration by the same route. An analysis of variance, ANOVA, followed by Fisher's protected least significant difference procedure for post hoc comparison, was used to verify significance between two means of hyperalgesic behavioral results from the Randall & Selitto apparatus paw pressure test (“anti-hyperalgesia”) or from the Von Frey test (“mechanical allodynia”) or from the Cold Plate test (“cold allodynia”). P values 0.05 were considered significant. This definition applies to any doses specified herein as inhibiting “in a statistically significant manner”.

### Paw Pressure Test (Hyperalgesia)

Paw mechanical sensitivity was determined using a Randall & Selitto apparatus (Randall and Selitto, 1957) exerting a force that increases at constant rate (32 g/s). The stimulus causing paw withdrawal was evaluated before and at different times after treatment. Results represent the mean of mechanical thresholds for paw withdrawal expressed as grams. To avoid possible damage to the rat paw the maximum applied force was set at 240 g. In the single administration protocol, paw pressure tests were performed before (pre-dose) and at regular intervals after treatment.

### Von Frey Test (Mechanical Allodynia)

Each animal was placed in a chamber with a mesh metal floor covered by a plastic dome that enabled the animal to walk freely, but not to jump. The mechanical stimulus was then delivered in the mid-plantar skin of left hind paw using an electronic von Frey apparatus (37400 Dynamic Plantar Aesthesiometer, Ugo Basile, Comerio, Varese, Italy). The cut-off was set at 50 g, while the increasing force rate (ramp duration) was set at 20 s. In the single administration protocol, pain threshold measurements have been performed before (pre-dose) and at regular intervals after treatment. In the repeated treatment protocol, the pre-test value was measured 13 h after the last administration; afterward the animals received a new administration and measures were performed at the described times. Results are expressed in grains and represent the mean±S.E.M. of mechanical thresholds.

### Cold Plate Test (Cold Allodynia)

The animals were placed in a stainless box (12 cm×20 cm×10 cm) with a cold plate as floor. The temperature of the cold plate was kept constant at 4° C.±1° C. Pain-related behaviors (i.e. lifting and licking of the hind paw) were observed and the time (s) of the first sign was recorded. The cut-off time of the latency of paw lifting or licking was set at 60 s (Di Cesare Mannelli et al. Exp Neurol 261:22-33, 2014).

### Example 2A: Effect of Compounds in the Chronic Constriction Injury Model

To evaluate the effect of test compounds in comparison to vehicle or selected comparators on mechanical hyperalgesia, the chronic constriction injury models in rats, a widely accepted model of painful neuropathy (Wang and Wang, 2003) was chosen.

Neuropathy was induced according to the procedure described by Bennett and Xie (1998). Briefly, rats were anaesthetized with chloral hydrate 400 mg/kg i.p. under aseptic conditions, the right common sciatic nerve was exposed at the level of the middle thigh by blunt dissection. Proximal to the trifurcation, the nerve was carefully freed from the surrounding connective tissue and four chromic cat gut ligatures (4-0, Ethicon, Norderstedt, Germany) were tied loosely around it with about 1 mm spacing. After hemostasis was confirmed, the incision was closed in layers. Then animals were allowed to recover from anesthesia and surgery and were kept one per cage with free access to water and standard laboratory chow. This procedure induces the appearance of hyperalgesia and allodynia in response to mechanical stimuli, which are evident 2-5 days after injury and reach their maximum severity in about 14 days.

TABLE 1
Mechanical antiallodynic effect of 2-substituted-sulfonyl-hexahydro-pyrrolo[1,2-
a]pyrazin-6(2H)-ones after oral administration to CCI rats

| | | | Lowest dose (mg/kg p.o.) at which CCI- | | |
|---|---|---|---|---|---|
| | | | induced mechanical allodynia is inhibited in | | |
| | | | a statistically significant manner (P < 0.05) | | |
| | | | Time after administration | | |
| | Substituent Z | Chirality | 30 min | 60 min | 90 min |
| NT-24336 | 2-fluorophenyl | R | 1 | 3 | 3 |
| NT-24337 | 3-fluorophenyl | R | 1 | 1 | 3 |

### Example 2B: Effect of Compounds in the MIA-Induced Osteoarthritis Model

### Knee Osteoarthritis Model

A single intra-articular injection of sodium monoiodoacetate (MIA) was introduced into the knee joint of rats according to the method described by Fernihough J et al., 2004. Sodium monoiodoacetate (MIA) inhibits chondrocyte metabolism leading to cartilage degradation in form of osteoarthritic-like focal lesions in the cartilage associated with subchondral bone thickening 14 days after administration (Guingamp et al., 1997). This model therefore can easily and quickly reproduce osteoarthritic-like lesions and functional impairment in rats, similar to that observed in human disease (Guzman et al., 2003). After 7 days post-injection, the inflammatory component subsides and the remaining pain is considered neuropathic in nature. Briefly, rats were deeply anaesthetized with diethyl ether. Following abolition of the hind paw pinch withdrawal reflex, a 27-gauge needle was introduced into the joint cavity between the tibial plateau and femoral condyles. Once in place, 2 mg of MIA were diluted in a volume of 25 mL of 1% CMC (carboxymethylcellulose in water, Sigma-Aldrich, Italy) and injected into one knee joint and the rat was allowed to recover for 14 days prior to pain assessment.

TABLE 2
Mechanical antihyperalgesic effect of 2-substituted-sulfonyl-hexahydro-pyrrolo[1,2-
a]pyrazin-6(2H)-ones after intravenous (i.v.) administration to MIA-treated rats

| | | | Lowest dose (mg/kg i.v.) at which MIA- | | |
|---|---|---|---|---|---|
| | | | induced mechanical hyperalgesia is inhibited | | |
| | | | in a statistically significant manner (P < 0.05) | | |
| | | | Time after administration | | |
| | Substituent Z | Chirality | 15 min | 30 min | 45 min |
| NT-24336 | 2-fluorophenyl | R | 3 | 3 | 3 |
| NT-24337 | 3-fluorophenyl | R | 3 | 3 | 3 |

### Example 2C. Diabetic Peripheral Neuropathy

Mice (=30 g) were injected intravenously in the tail with 200 mg/kg streptozotocin (Wako Pure Chemicals, Richmond, Va.) prepared in saline adjusted to pH 4.5 in 0.1 N citrate buffer. Age-matched non-diabetic control mice were injected with the vehicle alone. Streptozotocin solutions were freshly prepared due to the limited stability of the compound. Animals were kept in a group of four per cage with special care of food and water supplement. The bed of the cage was changed every day. In a set of preliminary control experiments, serum glucose level was measured spectrophotometrically at 7, 14, and 21 days after streptozotocin treatment and was found to be consistent with a diabetic level (above 300 mg/dl) throughout the periods. The serum glucose level was measured by glucose oxidase method from blood samples obtained by tail vein pricking. The animals were found to develop both thermal and mechanical hyperalgesia at 1st, 2nd, and 3rd weeks after streptozotocin treatment. Animals at 7 days post-streptozotocin treatment were used in the study.
The i.c.v. injections were carried out into the left lateral ventricle of mice. Injections were performed using a Hamilton microliter syringe fitted with a 26-gauge needle, according to the method of Haley and McCormick (1957). The site of injection was 2 mm caudal and 2 lateral to the bregma, and 3 mm in depth from the skull surface. The injection volume was 5 μl.

TABLE 3
Effect of two 2-substituted-sulfonyl-hexahydro-
pyrrolo[1,2-a]pyrazin-6(2H)-ones after oral
administration to streptozotocin-treated mice

| | | | Lowest dose (mg/kg p.o.) at | | | |
|---|---|---|---|---|---|---|
| | | | which streptozotocin-induced | | | |
| | | | diabetese-like thermal | | | |
| | | | hyperalgesia is inhibited in a | | | |
| | | | statistically significant manner | | | |
| | | | (P < 0.05) | | | |
| | | | Time after administration | | | |
| | | Chira- | 30 | 60 | 90 | 120 |
| | Substituent Z | lity | min | min | min | min |
| NT-24336 | 2-fluorophenyl | R | 30 | 30 | 30 | 30 |
| NT-24337 | 3-fluorophenyl | R | 10 | 10 | 10 | n.t. |

| | | | | |
|---|---|---|---|---|
| Duloxetine | 30 | 30 | 30 | 30 |

### Example 3A. Chemotherapy Induced Peripheral Sensor Neuropathy—Oxaliplatin Model

Peripheral sensory neuropathy was induced in adult rats, by administration of oxaliplatin (Tocris) at 2.4 m g i.p. in saline once daily for 5 consecutive days every week for three weeks (cumulative dose 36 mg/kg) according to Cavaletti et al., 2001. Starting from day 21 after the first oxaliplatin administration, the effect of repeated oral administration of racemic unifiram or the preferred inventive composition with an enantiomeric excess of (R)-unifiram of 50% corresponding to a 3:1 (R):(S) ratio of the enantiomers on oxaliplatin-induced mechanical hyperalgesia was assessed.

TABLE 4
Effect of NT-24336 after oral administration to oxaliplatin-treated rats

| | | Lowest dose (mg/kg p.o.) at which oxaliplatin-induced | | | |
|---|---|---|---|---|---|
| | | hyperalgesia is inhibited in a statistically significant | | | |
| | | manner (P < 0.05) | | | |
| | | Time after administration | | | |
| 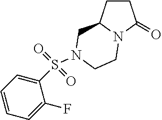 [SMILES: O=C1CC[C@@H]2CN(S(=O)(=O)c3ccccc3F)CCN12] | Chirality | 30 min | 60 min | 90 min | 120 min |
| NT-24336 | R | 1 | 1 | 1 | >10 |

### Example 3B. Chemotherapy Induced Peripheral Sensory Neuropathy—Vincristine Model

TABLE 5
Effect of NT-24336 after oral administration to vincristine-treated rats

| | | Lowest dose (mg/kg p.o.) at which vincristine-induced | | | |
|---|---|---|---|---|---|
| | | hyperalgesia is inhibited in a statistically significant | | | |
| | | manner (P < 0.05) | | | |
| | | Time after administration | | | |
| 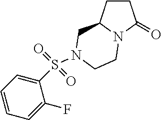 [SMILES: O=C1CC[C@@H]2CN(S(=O)(=O)c3ccccc3F)CCN12] | Chirality | 30 min | 60 min | 90 min | 120 min |
| NT-24336 | R | 10 | 10 | 10 | >10 |

### Example 3C. Chemotherapy Induced Peripheral Sensory Neuropathy—Paclitaxel Model

TABLE 6
Effect of some invention compounds after i.v. administration to paclitaxel-treated
rats

| | | Lowest dose (mg/kg i.v.) at which paclitaxel- | | | |
|---|---|---|---|---|---|
| | | induced hyperalgesia is inhibited in a statistically | | | |
| | | significant manner (P < 0.05) | | | |
| | | Time after administration | | | |
| 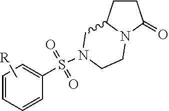 | Phenyl substituent (Chirality) | 15 min | 30 min | 45 min | 60 min |
| NT-24336 | 2-F (R) | 3 | 3 | 3 | n.t. |
| NT-24781 | 2-F (S) | >3 | >3 | >3 | n.t. |
| NT-24337 | 3-F (R) | 3 | 3 | 3 | n.t. |
| NT-24782 | 3-F (S) | >3 | >3 | >3 | n.t. |
| NT-24617 | 4-F (RS) | 3* | 3 | 3 | >3 |
| NT-24266 | 4-F (R) | 1 | 3 | >10 | n.t. |
| NT-24234 | 4-F (S) | 1 | 3 | >10 | n.t. |

*: NT-24617 was not tested at 1 mg/kg i.v.

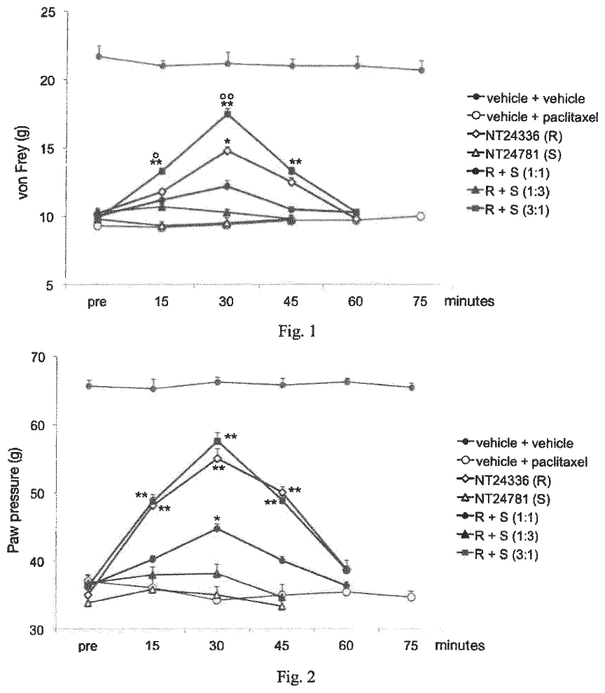

The efficacy of 2-(2-fluorobenzenesulfonyl)hexahydropyrrolo[1,2-a]pyrazin-6-one derivatives (R enantiomer=NT-24336, S-enantiomer=NT-24781, R:S mixtures 1:1, 1:3 and 3:1) was compared after a single acute administration (1 mg/kg i.v.) in a model of mechchanically induced hyperalgesia and allodynia in adult male Sprague Dawley rats.
Evaluation of mechanical allodynia (von Frey test) revealed that the non-racemic mixture containing three parts of the R-enantiomer and 1 part of S-enantiomer was, by far, the most active one, being more effective than the R-enantiomer in a statistically significant way (P<0.05 at 15 min after administration and P<0.01 after 30 min). The racemic 1:1 mixture was marginally effective whilst the S-enantiomer and the 1:3 mixture of R- and S-enantiomers were almost inactive (cf. FIG. 1). In the mechanical hyperalgesia (Randall & Selitto, paw pressure test) assay, the same rank order of potency was revealed (cf. FIG. 2).
FIG. 1 shows the effect on paclitaxel-induced mechanical allodynia of R- and S-enantiomers of 2-(2-fluorobenzenesulfonyl)hexahydropyrrolo[1,2-a]pyrazin-6-one and of different enantiomeric mixtures thereof. Paclitaxel (2 mg/kg i.p.) was administered on days 1, 3, 5 and 8. Test compounds were dissolved in saline and administered intravenously at the dose of 1 mg/kg to adult male Sprague-Dawley rats (Charles River, Italy, 220-250 g weight). Data were obtained at 0-75 minutes after injection and are expressed as the mean±s.e.m. of six animals. *P<0.05 and **<0.01 vs vehicle+paclitaxel treated rats; °P<0.05 and ^{°°}P<0.01 vs. NT24336 (R) treated group.
FIG. 2 shows the effect on paclitaxel-induced mechanical hyperalgesia of R- and S-enantiomers of 2-(2-fluorobenzenesulfonyl)hexahydropyrrolo[1,2-a]pyrazin-6-one and of different enantiomeric mixtures thereof. Paclitaxel (2 mg/kg i.p.) was administered on days 1, 3, 5 and 8. Test compounds were dissolved in saline and administered intravenously at the dose of 1 mg/kg to adult male Sprague-Dawley rats (Charles River, Italy, 220-250 g weight). Data were obtained at 0-75 minutes after injection and are expressed as the mean±s.e.m. of six animals. *P<0.05 and **<0.01 vss vehicle+paclitaxel treated rats.

### Example 4: In Vivo Anti-Amnestic Activity in a Passive Avoidance Paradigm in Mice

Racemic mixtures as well as R and S enantiomers were tested in a passive avoidance test in mice at doses of 3, 10 and 30 mg/kg, 30 min after oral administration.
Methods. The test was performed according to the step-through method described by Jarvik M E ad Kopp R, Psychol Rep, 21:221-224, 1967. The apparatus consisted of a two-compartment acrylic box, with a lighted compartment connected to a darkened one by a guillotine door. Mice receive a punishing electrical shock (0.3 mA, 1 s) as soon as they entered the dark compartment. The test was performed on two consecutive days. Mice were placed in the light side of the two-compartment box: the latency times for entering the dark compartment were measured in the training session on the first day, and after 24 h in the retention session on the second day. Mice received the punis, ent when entering the dark room in the training session and remembered it in the session on the following day, unless their memory was impaired by the amnesic drug. In the training session, mice which had not entered the dark compartment after 60 s latency were excluded from the remainder of the experiment; about 20-30% of mice were excluded from each group. All investigated drugs were administered orally 30 min prior to the training session; for memory disruption, mice were injected with the amnesic drug scopolamine (1.5 mg/kg i.p.) immediately after completion of the training session.
Vehicle-treated mice received an i.p. injection of saline immediately after the training session, as control of the scopolamine injection. After 24 h, the test was repeated (retention session); during the second day no drug was administered. The maximum entry latency allowed in the retention session was 180 s. The results are shown in Table 7.

TABLE 7
Antiamnestic effect of 2-phenylsulfonyl-hexahydro-pyrrolo[1,2-a]pyrazin-6(2H)-ones
after subcutaneous administration to scopolmine-treated mice*

| 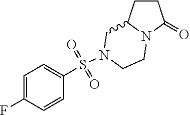 [SMILES: O=C1CCC2CN(S(=O)(=O)c3ccc(F)cc3)CCN12] | Chirality | Lowest Dose (μg/kg s.c.) at which scopolamine-induced amnesia is reversed in a statistically significant manner (P < 0.01) | Amnesia reversal, % |
|---|---|---|---|
| NT-24617 | racemic | 1 | 100 |
| NT-24266 | R | 1 | 75 |
| NT-24234 | S | 3 | 78 |

*: data from: Martini, E. et al., (2005). Medicinal Chemistry. 1(5): 473-480

The claimed ratios of R:S enantiomers of 2-phenylsulfonyl-hexahydro-pyrrolo[1,2-a]pyrazin-6(2H)-ones lead to more potent inhibition of glutamate release from rat spinal synaptosomes, more potent treatment of peripheral neuropathic pain, more potent anti-amnestic effects and more potent anti-depressant effects that are clearly better than in the case where the racemate of 2-phenylsulfonyl-hexahydro-pyrrolo[1,2-a]pyrazin-6(2H)-ones was used.
All patents, publications, and abstracts cited above are incorporated herein by reference in their entireties. Various embodiments of the invention have been described in fulfillment of the various objectives of the invention. It should be recognized that these embodiments are merely illustrative of the principles of the present invention. Numerous modifications and adaptions thereof will be readily apparent to those skilled in the art without departing from the spirit and scope of the present invention as defined in the following claims.

1. A composition comprising a compound of formula (I) and a compound of formula (II), 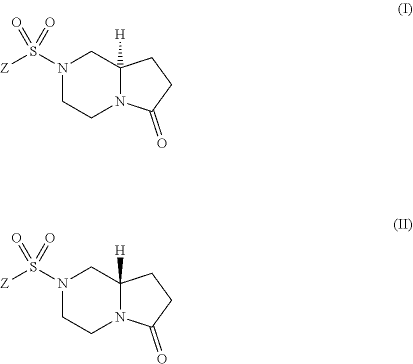
wherein Z is selected from
a straight chain, branched or cyclic C 1.4-alkyl group which is optionally substituted with one or more F,
or
a phenyl group which is substituted with R1 and R2, wherein R1 is selected from the group consisting of hydrogen, fluoro, chloro, cyano, trifluoromethyl and methyl; and R2 is independently selected from the group consisting of hydrogen, fluoro, chloro and methyl;
and R¹ and R² independently occupy any two positions on the phenyl ring;
and/or pharmaceutically acceptable solvates or co-crystals thereof,
wherein the enantiomeric excess (ee) of said compound of formula (I) is equal to or higher than 20% and lower than or equal to 50%.

2. The composition of claim 1, wherein the compound of formula (I) is a compound of formula (Ia) and the compound of formula (II) is a compound of formula (IIa), 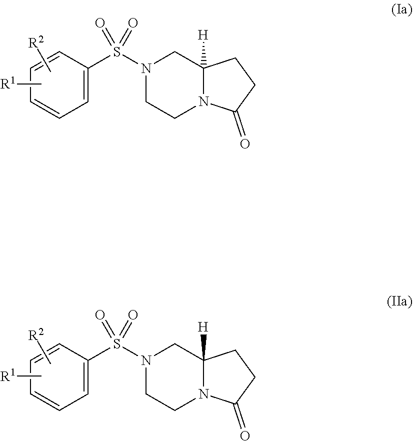
wherein R¹ is selected from the group consisting of hydrogen, fluoro, chloro, cyano, trifluoromethyl and methyl; and R² is independently selected from the group consisting of hydrogen, fluoro, cfaloro and methyl;
and R¹ and R² independently occupy any two positions on the phenyl ring;
and/or pharmaceutically acceptable solvates or co-crystals thereof,
wherein the enantiomeric excess (ee) of said compound of formula (Ia) is equal to or higher than 20% and lower than or equal to 50%.

3. The composition of claim 2, wherein the enantiomeric excess (ee) of said compound of formula (Ia) is equal to or higher than 30% and lower than or equal to 50%, preferably equal to or higher than 35% and lower than or equal to 50%.

4. The composition of claim 1, wherein the compound of formula (I) and/or pharmaceutically acceptable solvates or co-crystals thereof and the compound of formula (II) and/or pharmaceutically acceptable solvates or co-crystals thereof are packaged separately.

5. The composition of claim 1, wherein the composition is a non-racemic mixture of 2-([2-fluorophenyl]sulfonyl)-hexahydropyrrolo[1,2-a]pyrazin-6(2H)-one and pharmaceutically acceptable solvates or co-crystals thereof, wherein the non-racemic mixture comprises the compound of formula (I) and the compound of formula (II) in an enantiomeric excess (ee) of the compound of formula (I) of equal to or higher than 20% and lower than or equal to 50%.

6. The composition of claim 1, wherein the composition is a non-racemic mixture of 2-([4-fluorophenyl j sulfonyl)-hexahydropyrrolo [1,2-a]pyrazin-6(2H)˜one and pharmaceutically acceptable solvates or co-crystals thereof, wherein the non-racemic mixture comprises the compound of formula (I) and the compound of formula (II) in an enantiomeric excess (ee) of the compound of formula (I) of equal to or higher than 20% and lower than or equal to 50%.

7. The composition of claim 1, wherein the composition is a non-racemic mixture of 2-([3˜fluorophenyl]sulfonyl)-hexahydropyrrolo[1,2-aJpyrazin-6(2H)-one and pharmaceutically acceptable solvates or co-crystals thereof, wherein the non-racemic mixture comprises the compound of formula (I) and the compound of formula (II) in an enantiomeric excess (ee) of the compound of formula (I) of equal to or higher than 20% and lower than or equal to 50%

8. A pharmaceutical composition comprising the composition of claim 1 and a pharmaceutically acceptable carrier.

9. A kit of parts comprising a compound of formula (I) and a compound of formula (II) and instructions for combining the compound of formula (I) and the compound of formula (II) to obtain an enantiomeric excess (ee) of the compound of formula (I) of equal to or higher than 20% and lower than or equal to 50%.

10. (canceled)

11. A method of treating and/or preventing a disease, injury, or disorder in a patient, comprising administering to the patient the composition of claim 1, wherein the disease, injury or disorder is selected from the group consisting of peripheral sensory neuropathy, seizure, depression, anhedonia, a neuropsychiatric disorder, a motoneuron disorder, a movement disorder, and cognitive impairment.

12. (canceled)

13. The method of claim 11, wherein the peripheral sensory neuropathy is peripheral neuropathic pain.

14. The method of claim 11, wherein the disease, injury or disorder is depression or anhedonia, wherein preferably the depression or the anhedonia has not responded to previous treatment with established anti-depressant drugs.

15. The method of claim 11, wherein the peripheral sensory neuropathy is selected from the group consisting of diabetic neuropathy, post herpetic neuropathy, lumbago, sacral pain, surgical pain, crush injury, spinal injury, complex regional pain syndrome, phantom limb sensations, peripheral sensory neuropathy associated with osteoarthritis, peripheral sensory neuropathy associated with rheumatoid arthritis, peripheral sensory neuropathy associated with autoimmune osteoarthrosis, cephalea, fibromyalgia, peripheral sensory neuropathy induced by antiblastic therapies, peripheral sensory neuropathy induced by a chemotherapeutic agent, peripheral sensory neuropathy associated with visceral injury, peripheral sensory neuropathy associated with osteonecrosis, peripheral sensory neuropathy associated with human immunodeficiency virus infection, peripheral neuropathic pain, and peripheral sensory neuropathy induced by an antiviral agent.

16. The method of claim 11, wherein the peripheral sensory neuropathy is peripheral sensory neuropathy induced by a chemotherapeutic agent or peripheral sensory neuropathy induced by an antiviral agent.

17. The method of claim 16, wherein the peripheral sensory neuropathy is peripheral sensory neuropathy induced by a chemotherapeutic agent, wherein the chemotherapeutic agent is selected from the group consisting of a kinase inhibitor, a proteasome inhibitor, a taxane, a vinca alkaloid, and a platinum salt, and wherein preferably the chemotherapeutic agent is selected from sorafenib, sunitinib, afatinib, axitinib, vandetanib, vemurafenib, ixazomib, bortezomib, paclitaxel, docetaxel, cabazitaxel, vincristine, vinblastine, vmdesine, vinorelbine, nedaplatin, lobaplatin, picoplatin, satraplain, cisplatin, carboplatin and oxaliplatin.

18. The method of claim 16, wherein the peripheral sensory neuropathy is peripheral sensory neuropathy induced by an antiviral agent, wherein the antiviral agent is a nucleoside reverse transcriptase inhibitor, and wherein preferably the antiviral agent is selected from zalcitabine, didanosine, stavudine and zidovudine.

19. The method of claim 11, further comprising administering to the patient at least one antitumor drug, wherein preferably the antitumor drug is selected from the group consisting of a kinase inhibitor, a proteasome inhibitor, a taxane, a vinca alkaloid and a platinum salt; and wherein more preferably the antitumor drug is selected from sorafenib, sunitinib, afatinib, axitinib, vandetanib, vemurafenib, ixazomib, bortezomib, paclitaxel, docetaxel, cabazitaxel, vincristine, vinblastine, vindesine, vinorelbine, nedaplatin, lobaplatin, picoplatin, satraplain, cisplatin, carboplatin and oxaliplatin.

20. The method of claim 11, further comprising administering to the patient at least one antiviral drug, wherein preferably the antiviral drug is selected from a nucleoside or a nucleotide, and wherein further preferably the antiviral drug is selected from zalcitabine, didanosine, stavudine and zidovudine.

21. The method of claim 11, wherein the composition is administered orally twice daily in a dose of between 10 mg and 3000 mg per administration, more preferably between 20 mg to 2000 mg per administration, again more preferably between 50 mg and 1000 mg per administration.

22. A method to enhance learning and memory in a patient, the method comprising administering to the patient the composition of claim 1.

23. A method for preparing a composition of claim 1, comprising combining
a compound of formula (I), and a compound of formula (II), or
a compound of formula (I), and a racemate of a compound of formula (I) and (II).

24. (canceled)

25. The method of claim 22, wherein the patient is a healthy patient.
